(19) United States
(12) Patent Application Publication
(10) Pub. No.: US 2018/0142206 A1
(43) Pub. Date: May 24, 2018

(54) REVERSION OF PRIMED PLURIPOTENT STEM CELLS TO NAIVE PLURIPOTENT STEM CELLS

(71) Applicant: The J. David Gladstone Institutes, A Testamentary Trust Established under the will of J. David Glads, San Francisco, CA (US)

(72) Inventors: Cody Kime, San Francisco, CA (US); Kiichiro Tomoda, San Francisco, CA (US)

(21) Appl. No.: 15/571,556

(22) PCT Filed: May 4, 2016
(86) PCT No.: PCT/US16/30711

§ 371 Date: Nov. 3, 2017

Related U.S. Application Data
(60) Provisional application No. 62/255,990, filed on Nov. 16, 2015.
(60) Provisional application No. 62/157,411, filed on May 5, 2015.

(51) Int. Cl. C12N 5/0735; C12N 5/074

(52) U.S. Cl. CPC C12N 5/0606; C12N 5/0696; C12N 2500/38; C12N 2501/115; C12N 2501/155; C12N 2539/00; C12N 2501/235; C12N 2501/051; C12N 2320/31; C12N 2500/02; C12N 2501/16

(57) ABSTRACT

The present disclosure provides isolated naïve pluripotent stem cells as well as methods and compositions of generating and culturing the same.

### PRIORITY CLAIM

This application is a 371 U.S. National Stage application of International PCT Application No. PCT/US2016/030711, filed May 4, 2016, which claims priority to U.S. Provisional Application Ser. No. 62/157,411 which was filed on May 5, 2015, and to U.S. Provisional Application Ser. No. 62/255,990 which was filed on Nov. 16, 2015, their entire contents are incorporated herein by reference and relied upon.

### FIELD

The ability of pluripotent stem cells to differentiate into any cell type of the adult has generated much hope for the use of these cells—and cells derived from pluripotent stem cells—in the treatment of numerous diseases and disorders. One unresolved issue is the ability to generate and culture these cells in a more undifferentiated state known as the “naïve” state. These naïve pluripotent stem cells are believed to be most appropriate for use in regenerative therapies at least in part because the naïve state is associated with removal of epigenetic repressive markers and upregulation of pluripotency markers, which yields cells with fewer lineage and epigenetic restrictions. Thus, this disclosure provides novel methods and cell culture media for reverting and maintaining pluripotent stem cells in a naïve state.

### BACKGROUND

Two distinct pluripotent stem cell (PSC) states have been identified—a naïve and a primed state. These pluripotent states are distinguishable by molecular and cellular features. Naïve PSCs exhibit properties such as active X chromosome status, more relaxed chromatin, and self-renewal in response to Lif/Stat3, among others. On the other hand, primed PSCs exhibit properties such as inactive X chromosome, more condensed chromatin, and a lack of response to Lif/Stat3, among others. It is believed that naïve PSCs more closely resemble stem cells derived from the inner cell mass, while primed PSCs more closely resemble cells derived from the epiblast. In particular, human PSCs, including embryonic stem cells (ESCs) and induced pluripotent stem cells (iPSCs), share molecular and functional properties with epiblast stem cells (EpiSCs). Cell culture conditions can affect PSCs and cause PSCs, especially human PSCs, to exhibit features characteristic of a more primed PSC state. While both naïve and primed cells can differentiate into all three germ layers, naïve cells contribute to in vivo development more efficiently than primed cells.

Whether a true “naïve” state exists for human PSCs and whether suitable culturing techniques can be developed to revert PSCs and maintain a naïve state remains to be determined. Despite recent attempts, methods and media to derive naïve PSCs fail to either maintain self-renewal, preserve chromosome stability and/or are inefficient. A robust chemically-defined system to derive naïve PSCs remains unavailable. Such a system would likely result in a dramatic increase in the therapeutic potential of these cells. Thus, there remains a need for improved methods and media for the derivation and maintenance of naïve PSCs.

### SUMMARY OF THE DISCLOSURE

This disclosure is predicated on the discovery that a particular cocktail of agents can supplement a basal media and revert primed pluripotent stem cells to naïve pluripotent stem cells and is directed, in part, to methods of deriving a naïve pluripotent stem cell comprising: culturing a primed pluripotent stem cell in a culture media comprising an effective amount of an agonist of a lysophosphatidic acid receptor (LPAR), thereby reverting the primed pluripotent stem cell to a naïve pluripotent stem cell.

In other aspects, this disclosure provides methods for maintaining a naïve pluripotent stem cell in its naïve state, comprising culturing the naïve pluripotent stem cell in a culture media comprising an effective amount of an agonist of a LPAR.

In some aspects, this disclosure provides an isolated naïve pluripotent stem cell prepared by any of the methods disclosed and described herein.

In some aspects, this disclosure provides cell culture media for reverting a primed pluripotent stem cell into a naïve pluripotent stem cell comprising an agonist of a LPAR and wherein the primed pluripotent stem cell reverted to a naïve pluripotent stem cell during or after incubation. In some aspects, this disclosure provides a cell culture media comprising 10 nM to about 10 μM of an agonist of a LPAR. In yet other aspects, this disclosure provides cell culture media for culturing primed pluripotent stem cells comprising bFGF and further comprising one of more of activin A and TGF-β in a basal media.

In some aspects, this disclosure provides an isolated primed pluripotent stem cell, wherein the isolated primed pluripotent stem cell is incubated in the presence of a composition comprising an agonist of a LPAR and wherein the primed pluripotent stem cell reverted to a naïve pluripotent stem cell during or after incubation.

In some aspects, this disclosure provides an isolated naïve pluripotent stem cell prepared by a method comprising culturing the isolated naïve pluripotent stem cell in an effective amount of an agonist of a LPAR and wherein the primed pluripotent stem cell reverted to a naïve pluripotent stem cell during or after incubation. In another aspect, this disclosure provides, a composition comprising an isolated naïve pluripotent stem cell and an effective amount of an agonist of LPAR.

In one embodiment, the media further comprises an effective amount of one or more of a bone morphogenetic protein (BMP), an antioxidant, or a demethylase. In other embodiments, the media comprises a demethylase, an antioxidant, and an agonist of a LPAR. In yet other embodiments, the media comprises an effective amount of a demethylase and an agonist of a LPAR.

In one embodiment, the media further comprises an effective amount of a STAT3 activator, for example, leukemia inhibitor factor (LIF).

In some embodiments, the LPAR is selected from the group consisting of LPAR1, LPAR2, LPAR3, LPAR4, LPAR5 and LPAR6. In preferred embodiments, the LPAR is LPAR1. In some embodiments, the agonist of LPAR is OMPT. In other embodiments, the agonist of a LPAR is present in the media at a concentration from about 10 nM to about 10 μM. In other embodiments, the agonist of a LPAR is present in the media at a concentration of less than 5 μM.

In some embodiments, the antioxidant is n-acetyl cysteine. In some embodiments, the demethylase is ascorbic acid.

In some embodiments, the culture media further comprises an effective amount of at least one of an ERK1/2 inhibitor, GSK3β inhibitor, and a PKC inhibitor.

In some embodiments, the culture media further comprises an effective amount of activin A, transforming growth factor-β (TGF-β), bFGF, or any combination thereof.

In some embodiments, the naïve pluripotent stem cell is selected from the group consisting of an embryonic stem cell, a blastocyst, an induced pluripotent stem cell and a somatic cell. In some embodiments, the naïve pluripotent stem cell is a human cell.

In some embodiments, at least a portion of the isolated naïve pluripotent stem cells have been modified to express at least one exogenous reprogramming factor, at least one exogenous RNA, or both.

In some embodiments, the at least one exogenous reprogramming factor is Oct4, Sox2, L-Myc, C-Myc, Klf4, or Lin28.

In some embodiments, the at least one RNA down regulates expression of p53 or Lin28.

In some embodiments, the primed pluripotent stem cell and/or the naïve pluripotent stem cell is in hypoxic conditions. In other embodiments, the naïve pluripotent stem cell or the primed pluripotent stem cell is cultured in hypoxic conditions for a period of time

In some embodiments, the primed pluripotent stem cells and/or the naïve pluripotent stem cells are cultured on a fibronectin layer or a laminin layer.

In some embodiments, the primed pluripotent stem cell reverts to the naïve pluripotent stem cell in less than 10 days.

In some embodiments, greater than about 10% of the primed pluripotent stem cells revert to naïve pluripotent stem cells.

In some embodiments, the naïve pluripotent stem cell expressed XIST.

### BRIEF DESCRIPTION OF THE FIGURES

FIG. 1 demonstrates culture conditions that affect the X chromosome inactivation status in mouse epiblast stem cells (mEpiSCs). (A) Cell images showing cell morphology (Phase Contrast, PC) and GFP expression (GFP) of Xi-GFP mEpiSCs in three different culture conditions. Xi-GFP mEpiSCs are maintained in medium containing Activin A and bFGF (ActA+FGF) and are GFP negative (XaXi). After Xi-GFP mEpiSCs are transferred into medium containing LIF and two chemical inhibitors for MAPK and GSK3b (LIF+2i), or medium conditioned with SNL feeder cells (SNL-CM), some of Xi-GFP negative mEpiSCs become GFP positive (XaXa) in eight days. (B) A bar chart showing the number of GFP positive clusters in the three different media of panel A. (C) FACS dot plots showing GFP (x-axis) and CD31 (y-axis) expressions in (A). The % of GFP⁺CD31⁺ cells (upper right) are enlarged in the plots. (D) Bar chart showing relative numbers of GFP positive clusters in different culture conditions indicated. The number of GFP clusters in medium without conditioning (non-CM) is set as 1. “Jaki” indicates Jak kinase inhibitor, “MEF-CM” indicates medium conditioned with MEF feeder cells. (E) Bar chart showing concentrations of LIF in MEF-CM and SNL-CM quantified with ELISA. The concentration of recombinant LIF used in LIF+2i, non-CM+LIF and MEF-CM+LIF in (D) is also quantified and shown as LIF. LIF in MEF-CM is at undetectable levels by ELISA. (F) FACS dot plots showing GFP (x-axis) and CD31 (y-axis) expressions in the indicated medium conditions. ** shows p<0.01 and * shows p<0.05. Error bars are standard deviations of averaged values.

FIG. 2 demonstrates that Activin A and bFGF suppress LIF-induced Xi-reactivation. (A) Bar chart showing concentration of Activin A in MEF-CM and SNL-CM quantified with ELISA. (B) Bar chart showing relative numbers of GFP positive clusters in the different culture conditions indicated. (C) and (D) FACS dot plots showing GFP (x-axis) and CD31 (y-axis) expressions in the different culture conditions indicated. In (D), “ActR” indicates recombinant Activin A receptor protein complex, and “control” indicates control protein. (E) Scheme of the Activin A signaling pathway with respect to Xi-reactivation. (F) and (G) FACS dot plots showing GFP (x-axis) and CD31 (y-axis) expressions in the different culture conditions indicated. In (F), “SB” indicates SB431542, a chemical inhibitor of Smad2/3 activation. * shows p<0.05. Error bars are standard deviations of averaged values.

FIG. 3 demonstrates that ascorbic acid and LPA enhance Xi-reactivation. (A) Table showing components in the different culture media used in FIGS. 1 and 2. “AA” indicates ascorbic acid, and “Lipids” indicates lipid mixture in knock out serum replacement. (B) FACS dot plots showing GFP (x-axis) and CD31 (y-axis) expression in the different culture conditions indicated. (C) Bar chart showing fold changes in % of GFP⁺CD31⁺ cells in each culture condition indicated. Percentage of GFP⁺CD31⁺ cells in the LIF condition is set as 1. (D) FACS dot plots showing GFP (x-axis) and CD31 (y-axis) expression in the different culture conditions indicated. “Ki” indicates Ki16425, a competitive inhibitor of LPAR1 and LPAR3. (E) Bar chart showing fold changes in % of GFP⁺CD31⁺ cells in each culture condition indicated. Percentages of GFP⁺CD31⁺ cells in LIF or LIF+LPA condition without Ki16425 are set as 1. ** shows p<0.01 and * shows p<0.05. Error bars are standard deviations of averaged values.

FIG. 4 demonstrates that combining LIF, BMP4, Ascorbic Acid and OMPT (LBAO) efficiently reactivates Xi and converts primed pluripotent stem cells to naïve pluripotent stem cells. (A) FACS dot plots showing GFP (x-axis) and CD31 (y-axis) expression in medium containing LIF, BMP4, ascorbic acid and OMPT (LBAO). (B) FACS dot plots showing GFP⁺CD31⁺ cells selected from the culture shown in (A) by transferring cells into LIF+2i medium. Unstained (left) and stained samples (right) are shown. (C) RNA FISH for Atrx and Tsix in GFP⁺CD31⁺ cells. The % of cells which exhibit two nascent nuclear foci for both genes is inset. DNA is stained with DAPI. (D) Bar chart showing expression levels of pluripotency and differentiation markers relative to Gapdh in mESCs E14, GFP+CD31+ cells, and parental Xi-GFP mEpiSCs. Genes analyzed are indicated above the chart. The y-axis shows the expression levels of the genes at a log scale. (E) Images of embryos showing contribution of injected GFP⁺CD31⁺ cells to mouse development. The GFP positive embryo (bottom) indicates that GFP⁺CD31⁺ cells can contribute to the mouse development. The upper embryo is GFP negative, thus serving as a negative control. (F) Bar chart showing % of highly chimeric mice obtained by blastocyst injection of GFP⁺CD31⁺ cells or parental Xi-GFP mEpiSCs. Ten mice were obtained and analyzed after blastocyst injection of the GFP⁺CD31⁺ cells, while 30 mice were obtained and analyzed for the parental Xi-GFP mEpiSCs. No chimeras were obtained by the Xi-GFP mEpiSC injection. (G) Picture of F1 newborn pups showing germline transmission of blastocyst injected GFP⁺CD31⁺ cells. The lighter coat colored pups indicate the germline transmission.

FIG. 5 shows that LBAO orchestrates the transcription factors toward naïve pluripotency. (A) Line chart showing expression levels of the transcription factors, Klf2, Klf4, Prdm14 and Nanog, during the time course of the conversion in medium containing LIF, BMP4, ascorbic acid and OMPT (LBAO). The expression levels shown are relative to expression levels of Gapdh in parental Xi-GFP mEpiSCs (far left lane: EpiSCs) or at the indicated time points of the time course (from day2 to day6). (B) Line chart showing % of GFP⁺CD31⁺ (double), GFP⁺ (GFP single) and CD31⁺ cells (CD31 single) during the time course of the conversion (from day2 to day7). (C) Bar chart showing relative expression levels of Klf2, Klf4 and Nanog one day in the indicated medium (bottom of the chart). The expression levels in medium containing Activin A and bFGF (ActA+FGF) are set as 1. “Basal” indicates the same basal medium as ActA+FGF and LBAO but without adding any additional components. (D) Bar chart showing fold changes in % of GFP⁺ cells and expression levels of the transcription factors, Klf2 (blue), Klf4, Prdm14 and Nanog, cultured in the media indicated under the chart. The % of GFP⁺ cells and expression levels of the transcription factors in LBAO medium is set as 1. (E) Bar chart showing fold changes in % of GFP⁺ cells cultured in LBAO medium for 6 days after the knocking down of the genes of interest. The % of GFP⁺ cells after introducing MOCK vector is set as 1. The shRNA vectors that target each of the genes of interest and were introduced into Xi-GFP mEpiSCs are indicated under the bar chart. (F) Heat map showing relative expression levels of the genes analyzed (left of the heat map) in the shRNA expressing cells (above the heat map) examined in (F) six days in LBAO medium. The expression levels in GFP shRNA expressing cells are set as 1. The expression level indicator is shown on the right side of the heat map. ** shows p<0.01 and * shows p<0.05. Error bars are standard deviations of averaged values.

FIG. 6 shows that primed human induced pluripotent stem cells (hiPSCs) are converted to cells with characteristics of naïve PSCs in LPA-containing medium. (A) Comparison of colony morphology of hiPSCs in primed media and LPA containing media (reversion media). (b) Human iPSCs grown in reversion media stained with an antibody against NANOG (top left panel) and DNA (top right panel). Negative control shown in lower panels.

FIG. 7 shows that primed human induced pluripotent stem cells (hiPSCs) reprogrammed with non-integrating episomal vectors are converted to cells with characteristics of naïve PSCs when cultured in lipid containing medium. Panels show representative images of colony morphology of integration-free hiPSCs in OMPT- or OMPT+LPA-containing media as compared to control media containing no lipids. Culture in the lipid containing media converts the hiPSCs to cells with characteristics of naïve PSCs.

FIG. 8 shows cell morphology of primed PSCs cultured in various conditions of LPA and OMPT. Cell toxicities were observed in concentrations of LPA as low as 10 nM, while little to no toxicity was observed at high concentrations of OMPT (100 nM and 500 nM).

FIG. 9 shows naïve pluripotent stem cell marker expression in human naïve pluripotent stem cells versus primed pluripotent stem cells. Naïve cells expressed high levels of NANOG, KLF17, TFCP2L1 and KLF4 as compared to primed cells (NANOG and KLF17 data not shown for primed cells).

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of this disclosure will be limited only by the appended claims.

The detailed description of the disclosure is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a pluripotent stem cell” includes a plurality of pluripotent stem cells.

### I. Definitions

As used herein the following terms have the following meanings.

The term “about” when used before a numerical designation, e.g., temperature, time, amount, concentration, and such other, including a range, indicates approximations which may vary by (+) or (−) 10%, 5% or 1%.

Also as used herein, “and/or” refers to and encompasses any and all possible combinations of one or more of the associated listed items, as well as the lack of combinations when interpreted in the alternative (“or”).

As used herein, the term “agonist” refers to an agent, the presence of which results in an activity of a receptor that is the same as the activity resulting from the presence of a naturally occurring ligand for the receptor. An agonist of the lysophosphatidic acid receptor (LPAR) can bind to the LPAR and initiate a physiological or a pharmacological response characteristic of that receptor.

“Comprising” or “comprises” is intended to mean that the compositions, for example cell culture media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “stem cell” refers to a cell that is in an undifferentiated or partially differentiated state and has the capacity to self-renew and to generate differentiated progeny. Self-renewal is defined as the capability of a stem cell is to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells and hematopoietic stem cells. In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst); embryonic tissue; or fetal tissue taken any time during gestation, typically but not necessarily before approximately 10-12 weeks gestation. Most frequently, embryonic stem cells are totipotent cells derived from the early embryo or blastocyst. Embryonic stem cells can be obtained directly from suitable tissue, including, but not limited to human tissue, or from established embryonic cell lines.

The term “totipotent” refers to a stem cell that can give rise to any tissue or cell type in the body as well as extraembryonic tissue, such as the placenta. “Pluripotent” stem cells can give rise to any type of cell in the body. Stem cells that can give rise to a smaller or limited number of different cell types are generally termed “multipotent.” Thus, totipotent cells differentiate into pluripotent cells that can give rise to most, but not all, of the tissues necessary for fetal development. Pluripotent cells undergo further differentiation into multipotent cells that are committed to give rise to cells that have a particular function. For example, multipotent hematopoietic stem cells give rise to the red blood cells, white blood cells and platelets in the blood.

The term “pluripotent” as used herein refers to a cell with the capacity, under different conditions, to differentiate to cell types characteristic of all three germ cell layers (i.e., endoderm (e.g., gut tissue), mesoderm (e.g., blood, muscle, and vessels), and ectoderm (e.g., skin and nerve). Pluripotent cells are characterized primarily by their ability to differentiate to all three germ layers, using, for example, a nude mouse teratoma formation assay. Pluripotency is also evidenced by the expression of embryonic stem cell (ESC) markers, although the preferred test for pluripotency is the demonstration of the capacity to differentiate into cells of each of the three germ layers by, for example, an in vitro differentiation assay. Two phases of pluripotency can exist, namely, a naïve state and a primed state.

As used herein, the term “naïve state” in reference to a pluripotent stem cell refers to cell typically identified by the following characteristics: expresses high levels of the pluripotency factors Oct4, Nanog, Sox2, Klf2 and Klf4; self-renew in response to either Lif/Stat3 or 2i (ERKi/GSKi); differentiate in response to Fgf/Erk; exhibit a XaXa X-chromosome status; among others. Nichols et al., (2009) Cell Stem Cell 4(6):487-492. It is suggested that naïve cells contribute to in vivo development more efficiently than primed cells and are therefore thought to be the more appropriate cell type for regenerative medicine. The naïve state is sometimes referred to as the ground state. The term “naïve-like” in reference to a stem cell refers to a cell that expresses at least one of the characteristics of the naïve state. In some instances a naïve-like stem cell expresses at least one characteristic of the naïve state at a degree that is less than that expressed by a naïve stem cell.

As used herein, the term “primed state” in reference to a pluripotent stem cell refers to a cell typically identified by the following characteristics: expresses high levels of pluripotency factors Oct4, Sox2 and Nanog; do not respond to Lif/Stat3; self-renew in response to Fgf/Erk; exhibit a XaXi X-chromosome activation status; among others. Nichols et al., (2009) Cell Stem Cell 4(6):487-492.

As used herein the term “isolated” with reference to a cell, refers to a cell that is, at least partially, in an environment different from that in which the cell naturally occurs, e.g., where the cell naturally occurs in a multicellular organism, and the cell is removed from the multicellular organism, the cell is “isolated.” For example, an isolated cell is a cell that is separated form tissue or cells of dissimilar phenotype or genotype. As is apparent to those of skill in the art, a non-naturally occurring polynucleotide, peptide, polypeptide, protein, does not require “isolation” to distinguish it from its naturally occurring counterpart.

The term “induced pluripotent stem cells” shall be given its ordinary meaning and shall also refer to differentiated mammalian somatic cells (e.g., adult somatic cells, such as skin) that have been reprogrammed to exhibit at least one characteristic of pluripotency. Induced pluripotent stem cells (iPSCs) can be derived using any method known in the field, such as, retroviral, lentiviral, episomal, small molecule, or modified RNAs. See, for example, Takahashi et al. (2007) Cell 131(5):861-872, Kim et al. (2011) Proc. Natl. Acad. Sci. 108(19): 7838-7843, Sell, S. Stem Cells Handbook. New York: Springer, 2013. Print. Warren et al. (2010) Cell Stem Cell 7(5):618-630.

As used herein the terms “culture media” and “culture medium” are used interchangeably and refer to a solid or a liquid substance used to support the growth of cells (e.g., stem cells). Preferably, the culture media as used herein refers to a liquid substance capable of maintaining stem cells in an undifferentiated state. The culture media can be a water-based media which includes a combination of ingredients such as salts, nutrients, minerals, vitamins, amino acids, nucleic acids, proteins such as cytokines, growth factors and hormones, all of which are needed for cell proliferation and are capable of maintaining stem cells in an undifferentiated state. For example, a culture media can be a synthetic basal media such as, for example, DMEM/F12, GlutaMAX (Life Technologies, Carlsbad, Calif., USA), Neurobasal Medium (Life Technologies, Carlsbad, Calif., USA), KO-DMEM (Life Technologies, Carlsbad, Calif., USA), DMEM/F12 (Life Technologies, Carlsbad, Calif., USA), supplemented with the necessary additives as is further described herein. In some embodiments, the culture media can be a culture media formulated specifically for use with stem cells, for example, mTeSR (Stem Cell Technologies, Vancouver, BC, Canada) or StemFit (Ajinomoto Co., Tokyo, JP). In some embodiments, the cell culture media can be a mixture of culture media. Preferably, all ingredients included in the culture media of the present disclosure are substantially pure and tissue culture grade.

As used herein the terms “basal media” and “basal medium,” are used interchangeably, and refer a culture media without additives. Basal media can include a mixture of different basal media.

As used herein the term “equivalents thereof” refers to an agent with the same or similar function and/or the same or similar ingredients. An equivalent thereof might be used as an alternative agent by those skilled in the art. For example, an equivalent thereof of Neurobasal medium may be NeuralQ medium (Sigma-Aldrich, St. Louis, Mo., USA). An equivalent thereof may also be a homemade version of a commercially available medium. For example, an equivalent of DMEM-F12-Glutamax could be made by combining 2.5 mM L-glutamine (or Glutamax), 15 mM HEPES, 0.5 mM sodium pyruvate, and 1200 mg/L sodium bicarbonate.

An “effective amount” is an amount of an agent or compound (e.g., BMP4, exogenous RNA, agonist of LPAR) sufficient to effect beneficial or desired results. An effective amount can be in one or more administrations, applications or dosages. Determination of these parameters is well within the skill of the art. These considerations, as well as effective formulations and administration procedures are well known in the art and are described in standard textbooks.

### II. Cells

This invention is predicated on the discovery that a particular cocktail of agents can supplement a basal media and revert primed pluripotent stem cells to naïve pluripotent stem cells (PSCs).

The PSCs of the present invention include any pluripotent cell, including for example, an embryonic stem cell (ESC), an embryonic germ cell (EGC), a blastocyst, an induced pluripotent stem cell (iPSC) or a somatic cell. In some embodiments, the naïve pluripotent stem cell is selected from the group consisting of an ESC, an EGC, a blastocyst, an iPSC or a somatic cell. In other embodiments, the primed pluripotent stem cell is selected from the group consisting of an ESC, an EGC, a blastocyst, an iPSC or a somatic cell.

The PSCs of the present invention may be derived from a mammal, preferably a human, but includes and is not limited to non-human primates, murines (i.e., mice and rats), canines, felines, equines, bovines, ovines, porcines, caprines, etc. In some embodiments, the PSC is a human PSC.

ESCs can be obtained by any method known in the field. For example, ESCs can be isolated from a blastocyst-stage or epiblast-stage embryo. ESCs have been obtained from many species, including, but limited to, mouse (Mills et al. (2001) Trends Genet. 17:331-339); rat (Iannaccone et al. (1994) Dev. Biol. 163:288-292); non-human primate (Thomson et al. (1995) Proc. Natl. Acad. Sci. USA 92:7844-7848); and human (Thomson et al., (1998) Science 282(5391):1145-1147). Human ESCs (hESCs) are typically obtained from leftover or discarded human in vitro fertilization (IVF) embryos, but can be obtained from any available source and by any method known in the field. For example, some methods involve removing a single cell from an embryo during the IVF process. Chung et al. (2008) Cell Stem Cell 2(2):113-117. This method allows for the generation of a hESC line without concomitant destruction of the embryo. It will also be appreciated that many commercially available hESCs are available and can be used according to the embodiments of the disclosure. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), WA15, HUES 62, HUES 63, HUES 64, CT1, CT2, CT3, CT4, MA135, Eneavour-2, WIBR1, WIBR2, WIBR3, WIBR4, WIBR5, WIBR6, HUES 45, Shef 3, Shef 6, BJNhem19, BJNhem20, SA001, SA001, UCLA 10, UCLA 17, UCLA 18, HS346, HS420, NYUES12, KCL023, KCL031, UM63-1, UM77-2, CSC14, HUES 74, and UM25-2.

Embryonic germ cells (EGCs) are derived from germ cells (gametes) that, under special conditions, acquire properties similar to those of ESCs. Like ESCs, EGCs are pluripotent in vitro; however EGCs have yet to be shown to be pluripotent in vivo. EGCs are prepared from the primordial gem cells obtained from fetuses using techniques well-known in the art. Shamblott et al. (1998) Proc. Natl. Acad. Sci USA 95:13726-13731.

Induced pluripotent stem cells (iPSCs) can be generated from numerous types of somatic cells (e.g. fibroblasts, hepatocytes, epithelial cells) by either genetic (e.g., retroviral, adenoviral, episomal), protein, modified RNAs, or chemical manipulation to promote the expression of factors such as Oct4, Sox2, c-Myc, Klf4 (Yamanaka et al. (2006) Cell 126(4):663-676; Yamanaka et al. (2007) Cell Stem Cell 1(1):39-49; Takahashi et al. (2007) Cell 131:861-872).

As will be apparent to the skilled artisan upon reading this disclosure, the present disclosure provides an isolated naïve PSC (e.g., ESC, iPSC, and the like). A naïve PSC can be identified as having at least some of the following characteristics: expresses high levels of the pluripotency factors Oct4, Nanog, Sox2, Klf2 and Klf4; self-renew in response to either Lif/Stat3 or 2i (ERKi/GSKi); differentiate in response to Fgf/Erk; exhibit a XaXa X-chromosome activation status; among others. Nichols et al., (2009) Cell Stem Cell 4(6):487-492; Weinberger et al., (2016) Nature Reviews 17:155-168. In some embodiments, at least one characteristic typical of a naïve PSC is expressly excluded.

As will be apparent to the skilled artisan upon reading this disclosure, the present disclosure provides an isolated primed PSC (e.g., ESC, iPSC, and the like). A primed PSC can be identified as having at least some of the following characteristics: expresses high levels of pluripotency factors Oct4, Sox2 and Nanog; do not respond to Lif/Stat3; self-renew in response to Fgf/Erk; exhibit a XaXi X-chromosome activation status; among others. Nichols et al., (2009) Cell Stem Cell 4(6):487-492; Weinberger et al., (2016) Nature Reviews 17:155-168. In some embodiments, at least one characteristic typical of a primed PSC is expressly excluded.

In some embodiments, the PSCs of the present disclosure are genetically modified. In preferred embodiments, the PSCs have not been modified to inhibit MEK, GSK3, PKC, or any combination thereof.

In some embodiments, the naïve and/or primed PSCs of the present disclosure express at least one exogenous reprogramming factor, at least one exogenous RNA, or both. The exogenous reprogramming factor can be any reprogramming factor known to one skilled in the art. Non-limiting examples of reprogramming factors include Oct4, Sox2, L-Myc, C-Myc, Nanog, Klf4, and Lin28. In some embodiments, the reprogramming factors are expressed by a vector, for example, an episomal vector, a retroviral vector, a lentiviral vector, or a Sendai viral vector. In some embodiments, the exogenous RNA may be, for example, short interfering RNA (siRNA), microRNA (miRNA), short hairpin RNA (shRNA) and modified RNA (mRNA). The exogenous RNA may bind to any target known to one skilled in the art. Non-limiting examples of targets include p53, Lin28, Klf2, Prdm14, and Nanog. In some embodiments, the exogenous RNA down regulates expression of p53 or Lin28. In one preferred embodiment, the naïve and/or primed cells have not been modified to express at least one exogenous reprogramming factor.

The primed and naïve PSCs of the present disclosure can be cultured in any combination of the media and conditions or by any of the methods described in detail below.

### III. Cell Culture Media and Culture Conditions

### Reversion Media

As will be apparent to the skilled artisan upon reading this disclosure, the present disclosure provides cell culture media for reverting a primed pluripotent stem cell into a naïve pluripotent stem cell and/or maintaining a PSC in a naïve state. As used herein, the cell culture media for reverting a primed PSC into a naïve PSC and/or maintaining a PSC in a naïve state may be referred to as “reversion media.”

In some embodiments, the reversion media comprises a basal media. The basal media can be any basal media known to those in the art. The basal media can be a mixture of different basal media. The basal media may be purchased from commercial sources. In some embodiments, the basal medium of the reversion media comprises at least one of DMEM/F12-Glutamax, Neurobasal medium, N2 supplement (Life Technologies, Carlsbad, Calif., USA), B27 supplement (Life Technologies, Carlsbad, Calif., USA), BSA Fraction V (Life Technologies, Carlsbad, Calif., USA), and Glutamax (Life Technologies, Carlsbad, Calif., USA), or equivalents thereof. In some embodiments, the basal medium of the reversion media comprises each of DMEM/F12-Glutamax, Neurobasal medium, N2 supplement (Life Technologies, Carlsbad, Calif., USA), B27 supplement (Life Technologies, Carlsbad, Calif., USA), BSA Fraction V (Life Technologies, Carlsbad, Calif., USA), and Glutamax (Life Technologies, Carlsbad, Calif., USA), mTeSR (Stem Cell Technologies, Vancouver, BC, Canada) or StemFit (Ajinomoto Co., Tokyo, JP) or equivalents thereof.

In some embodiments, the DMEM/F12-Glutamax or equivalent thereof, is present in the reversion media at between about 25% and about 75% of the volume of the reversion media. In preferred embodiments, the DMEM/F12-Glutamax or equivalent thereof, is present in the reversion media at about 50% of the volume of the reversion media.

In some embodiments, the Neurobasal medium or equivalent thereof, is present in the reversion media at between about 25% and about 75% of the volume of the reversion media. In preferred embodiments, the Neurobasal medium or equivalent thereof is present in the reversion media at about 50% of the volume of the reversion media.

In some embodiments, the N2 supplement or equivalent thereof is present in the reversion media at between about 0.0005% and about 0.05% of the volume of the reversion media. In preferred embodiments, the N2 supplement or equivalent thereof is present in the reversion media at about 0.005% of the volume of the reversion media.

In some embodiments, the B27 supplement or equivalent thereof is present in the reversion media at between about 0.001% and about 0.1% of the volume of the reversion media. In preferred embodiments, the B27 supplement or equivalent thereof is present in the reversion media at about 0.01% of the volume of the reversion media.

In some embodiments, the BSA Fraction V or equivalent thereof is present in the reversion media at between about 0.00007% and about 0.007% (from a 7.5% solution) of the volume of the reversion media. In preferred embodiments, the BSA Fraction V or equivalent thereof is present in the reversion media at about 0.0007% (from a 7.5% solution) of the volume of the reversion media.

In some embodiments, the Glutamax or equivalent thereof is present in the reversion media at between about 0.0005% and about 0.05% of the volume of the reversion media. In preferred embodiments, the Glutamax or equivalent thereof is present in the reversion media at about 0.005% of the volume of the reversion media.

In some embodiments, the reversion media comprises a basal media and further comprises at least one additive (i.e., agent). In some embodiments the at least one additive is, for example, an agonist of a lysophosphatidic acid receptor (LPAR), bone morphogenetic protein (BMP), a demethylase, a STAT3 activator, 2-mercaptoethanol (2-ME), bFGF, Activin A, TGFβ, a MEK inhibitor, a GSK3β inhibitor, a PKC inhibitor, forskolin, a ROCK inhibitor, or a JNK inhibitor. In some embodiments, the reversion media comprises a basal media and further comprises each of an agonist of a lysophosphatidic acid receptor (LPAR), bone morphogenetic protein (BMP), a demethylase, a STAT3 activator, and 2-mercaptoethanol (2-ME). In some embodiments, the reversion media comprises a basal media and further comprises each of an agonist of a lysophosphatidic acid receptor (LPAR), a demethylase, a STAT3 activator, and 2-mercaptoethanol (2-ME). In some embodiments, the reversion media comprises a basal media and further comprises each of an agonist of a lysophosphatidic acid receptor (LPAR), a demethylase, a STAT3 activator, and Activin A (and/or TGFβ). In some embodiments, the reversion media comprises a basal media and further comprises each of an agonist of a lysophosphatidic acid receptor (LPAR), a demethylase, a STAT3 activator, Activin A (and/or TGFβ) and bFGF. In one preferred embodiment, the reversion media comprises a basal media (e.g., StemFit), Activin A (and/or TGFβ), a MEK inhibitor, a GSK3β inhibitor, an LPAR (e.g., OMPT or LPA), N-Acetyl Cysteine, and optionally LIF and/or bFGF. In a preferred embodiment, forskolin is expressly excluded.

In some embodiments, the reversion media comprises a basal media and an agonist of a LPAR. In some embodiments, the LPAR is selected from the group consisting of LPAR1, LPAR2, LPAR3, LPAR4, LPAR5 and LPAR6. In preferred embodiments, the LPAR is LPAR1. In some embodiments the agonist of a LPAR is present in the media at a concentration from about 10 nM to about 5 μM. It is contemplated that the agonist of a LPAR may function, at least in part, by activating Yes-associated protein (YAP) transcription factors. Thus, it is also contemplated that activating YAP with other small molecules or inhibiting pathways that sequester YAP to the cytoplasm (e.g. Lats kinases) may also be added to the reversion media. A non-limiting example of an activator of YAP includes sphingosine-1-phosphate. Miller et al. (2012) Chem Biol 19(8):955-962 and Yu et al. (2012) Cell 150:780-791.

In some embodiments, the agonist of a LPAR is lysophosphatidic acid (LPA). The LPA of the present disclosure can be at a concentration in the reversion media, for example, from about 10 nM to about 1 μM, from about 100 nM to about 1 μM, from about 200 nM to about 1 μM, from about 300 nM to about 1 μM, from about 400 nM to about 1 μM, from about 500 nM to about 1 μM, from about 600 nM to about 1 μM, from about 700 nM to about 1 μM, from about 800 nM to about 1 μM, from about 900 nM to about 1 μM, from about 10 nM to about 100 nM, from about 50 nM to about 100 nM, from about 60 nM to about 100 nM, from about 70 nM to about 100 nM, from about 80 nM to about 100 nM, from about 90 nM to about 100 nM, about 10 nM, about 20 nM, about 30 nM, about 40 nM, about 50 nM, about 60 nM, about 70 nM, about 80 nM, about 90 nM, about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, or about 1 μM. In preferred embodiments, the amount of LPA is less than 10 μM. In more preferred embodiments, the amount of LPA is less than 5 μM. In some embodiments, a LPA is expressly excluded from the reversion media.

In some embodiments, the agonist of a LPAR is 1-oleoyl-2-methyl-sn-glycero-3-phosphothioate (OMPT). The OMPT of the present disclosure can be at a concentration in the reversion media, for example, from about 0.1 μM to about 10 μM, from about 0.5 μM to about 10 μM, from about 1.0 μM to about 10 μM, from about 2.0 μM to about 10 μM, from about 3.0 μM to about 10 μM, from about 4.0 μM to about 10 μM, from about 5.0 μM to about 10 μM, from about 6.0 μM to about 10 μM, from about 7.0 μM to about 10 μM, from about 8.0 μM to about 10 μM, from about 9.0 μM to about 10 μM, about 0.5 μM, about 0.6 μM, about 0.7 μM, about 0.8 μM, about 0.9 μM, about 1.0 μM, about 2.0 μM, about 3.0 μM, about 4.0 μM, about 5.0 μM, about 6.0 μM, about 7.0 μM, about 8.0 μM, about 9.0 μM, or about 10 μM.

In some embodiments, the agonist of a LPAR is a combination of at least two agonists of a LPAR. In one embodiment, the reversion media comprises a combination of an amount of OMPT and LPA.

In some embodiments, the reversion media comprises a basal media and at least one bone morphogenetic protein (BMP), for example, BMP1, BMP2, BMP3, BMP4, BMP5, BMP6, BMP7, BMP8a, BMP8b, BMP10, and BMP15. In preferred embodiments, the BMP is BMP4. The BMP of the present disclosure can be at a concentration in the reversion media, for example, from about 0.1 ng/mL to about 100 ng/mL, from about 0.5 ng/mL to about 50 ng/mL, from about 1.0 ng/mL to about 25 ng/mL, from about 2.5 ng/mL to about 12.5 ng/mL, from about 5 ng/mL to about 10 ng/mL, from about 0.5 ng/mL to about 15 ng/mL, from about 0.6 ng/mL to about 12 ng/mL, from about 0.8 ng/mL to about 11 ng/mL, from about 0.9 ng/mL to about 10 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 15 ng/mL, about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL. In some embodiments, a BMP is expressly excluded from the reversion media.

In some embodiments, the reversion media comprises a basal media and at least one demethylase, for example, ascorbic acid (e.g., L-ascorbic acid and ascorbic acid-2 phosphate). The demethylase (e.g. ascorbic acid) of the present disclosure can be at a concentration in the reversion media, for example, from about 1 μg/mL to about 200 μg/mL, from about 10 μg/mL to about 150 μg/mL, from about 20 μg/mL to about 100 μg/mL, from about 30 μg/mL to about 90 μg/mL, from about 40 μg/mL to about 80 μg/mL, from about 50 μg/mL to about 70 μg/mL, from about 55 μg/mL to about 65 μg/mL, about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, or about 100 μg/mL.

In some embodiments, the reversion media comprises a basal media and at least one antioxidant, for example, N-Acetyl Cysteine (NAC). The antioxidant of the present disclosure can be at a concentration in the reversion media, for example, from about 0.1 mM to about 10 mM, from about 0.5 mM to about 10 mM, from about 1.0 mM to about 10 mM, from about 2.0 mM to about 10 mM, from about 3.0 mM to about 10 mM, from about 4.0 mM to about 10 mM, from about 5.0 mM to about 10 mM, from about 6.0 mM to about 10 mM, from about 7.0 mM to about 10 mM, from about 8.0 mM to about 10 mM, from about 9.0 mM to about 10 mM, about 0.5 mM, about 0.6 mM, about 0.7 mM, about 0.8 mM, about 0.9 mM, about 1.0 mM, about 1.5 mM, about 2.0 mM, about 2.5 mM, about 3.0 mM, about 3.5 mM, about 4.0 mM, about 4.5 mM, about 5.0 μM, about 6.0 μM, about 7.0 μM, about 8.0 μM, about 9.0 μM, or about 10 μM. In one preferred embodiment, the antioxidant is at a concentration in the reversion media of about 2 mM.

In some embodiments, the reversion media comprises a basal media and a STAT3 activator, for example, leukemia inhibitor factor (LIF). The STAT3 (e.g. LIF) of the present disclosure can be at a concentration in the reversion media, for example, from about 100 units to about 10,000 units, 300 units to about 5,000 units, 500 units to about 4,000 units, 700 units to about 3,000 units, 900 units to about 2,000 units, 950 units to about 1,500 units, about 100 units, about 200 units, about 300 units, about 400 units, about 500 units, about 600 units, about 700 units, about 800 units, about 900 units, about 1,000 units, about 1,100 units, about 1,200 units, about 1,300 units, about 1,400 units, about 1,500 units, about 1,600 units, about 1,700 units, about 1,800 units, about 1,900 units, about 2,000 units, about 3,000 units, about 4,000 units, about 5,000 units, about 6,000 units, about 7,000 units, about 8,000 units, about 9,000 units, or about 10,000 units.

In some embodiments, the reversion media comprises a basal media and beta-mercaptoethanol (2-ME). The 2-ME (1000×) of the present disclosure can be at a concentration in the reversion media, for example, from about 0.0001% to about 0.01%. In preferred embodiments, the 2-ME is at a concentration in the reversion media of about 0.001%.

In some embodiments, the reversion media comprises a basal media and bFGF. The bFGF of the present disclosure can be at a concentration in the reversion media, for example, from about 1 ng/mL to about 25 ng/mL, about 5 ng/mL to about 20 ng/mL, about 10 ng/mL to about 15 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, or about 25 ng/mL.

In some embodiments, the reversion media comprises a basal media and Activin A. The Activin A of the present disclosure can be at a concentration in the reversion media, for example, from about 5 ng/mL to about 50 ng/mL, about 10 ng/mL to about 40 ng/mL, about 15 ng/mL to about 30 ng/ml, about 15 ng/mL to about 25 ng/ml, about 5 ng/mL, about 10 ng/mL, about 15 ng/mL, about 20 ng/mL, about 25 ng/mL, about 30 ng/mL, about 35 ng/mL, about 40 ng/mL, or about 50 ng/mL.

In some embodiments, the reversion media comprises a basal media and TGF-β. The TGF-β of the present disclosure can be at a concentration in the reversion media, for example, from about 0.1 ng/mL to about 10 ng/mL, about 0.5 ng/mL to about 5 ng/mL, about 1 ng/mL to about 4 ng/mL, about 2 ng/mL to about 3 ng/mL, about 0.1 ng/mL, about 0.2 ng/mL, about 0.3 ng/mL, about 0.4 ng/mL, about 0.5 ng/mL, about 0.6 ng/mL, about 0.7 ng/mL, about 0.8 ng/mL, about 0.9 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, or about 10 ng/mL.

In some embodiments, the reversion media comprises an effective amount of at least one of an ERK1/2 inhibitor, GSK3β inhibitor, and a protein kinase C (PKC) inhibitor. In some embodiments, the reversion media further comprises an ERK1/2 inhibitor (i.e., MEK inhibitor), a GSK3β inhibitor, or both. Use of ERK1/2 and GSK3β inhibitors for reversion have been described previously in, for example, Gafni et al., (2013) Nature 504(7479):282-286; Chan et al., (2013) Cell Stem Cell 12(6):663-675; Valamehr et al., (2014) Stem Cell Reports 2(3):366-381; Ware et al., (2014) Proc Natl Acad Sci USA 111(12):4484-4489; Theunissen et al., (2014) Cell Stem Cell 15(4):471-187. Non-limiting examples of MEK inhibitors include selumetinib (i.e. AZD6244), PD0325901, trametinib (GSK1120212), U0126-EtOH, PD184352 (CI-1040), GDC-0623, BI-847325, cobimetinib (GDC-0973, RG7420), PD98059, BIX 02189, binimetinib, pimasertib, SL-327, BIX 02188, AZD8330, TAK-733, Honokiol, PD318088, and refametinib. Non-limiting examples of GSK3β inhibitors include CHIR99021, GSK-3 inhibitor IX, lithium chloride, CHIR98014, GSK-3β inhibitor VI, 3F8, and TCS 2002. In one embodiment, the MEK inhibitor is PD98059. In another embodiment, the GSK3β inhibitor is CHIR99021. In one embodiment, the reversion media is substantially free of ERK1/2 inhibitor, GSK3β inhibitor, or both. In some embodiments, the reversion media is substantially free media is substantially free of activin A, bFGF, or both.

Protein kinase C (PKC) inhibitors are well known in the art and commercially available. Non-limiting examples include Go 6983 (Sigma-Aldrich, St. Louis, Mo., USA), Go 6976 (Tocris, Bristol, UK), Chelerythrine chloride (Tocris, Bristol, UK), HA-100 dihydrochloride (Santa Cruz Biotechnology, Dallas, Tex., USA). The PKC inhibitors (e.g. Gö 6983) of the present disclosure can be at a concentration in the reversion media, for example, from about 0.1 μM to about 10 μM, from about 0.5 μM to about 10 μM, from about 1.0 μM to about 10 μM, from about 2.0 μM to about 10 μM, from about 3.0 μM to about 10 μM, from about 4.0 μM to about 10 μM, from about 5.0 μM to about 10 μM, from about 6.0 μM to about 10 μM, from about 7.0 μM to about 10 μM, from about 8.0 μM to about 10 μM, from about 9.0 μM to about 10 μM, about 0.5 μM, about 0.6 μM, about 0.7 μM, about 0.8 μM, about 0.9 μM, about 1.0 μM, about 2.0 μM, about 3.0 μM, about 4.0 μM, about 5.0 μM, about 6.0 μM, about 7.0 μM, about 8.0 μM, about 9.0 μM, or about 10 μM.

In some embodiments, the reversion media comprises the media further comprises at least one of an activin A inhibitor and a transforming growth factor-β (TGF-β) inhibitor. Non-limiting examples include SB-431542 (Tocris, Bristol, UK), follistatin-344 (R&D Systems, Minneapolis, Minn., USA), SB525334 (Tocris, Bristol, UK), SB505124 (Tocris, Bristol, UK), and RepSox (Tocris, Bristol, UK). The activin A inhibitor, TGF-β inhibitor, or both of the present disclosure (e.g. SB-431542) can be at a concentration in the reversion media, for example, from about 0.1 μM to about 10 μM, from about 0.5 μM to about 10 μM, from about 1.0 μM to about 10 μM, from about 2.0 μM to about 10 μM, from about 3.0 μM to about 10 μM, from about 4.0 μM to about 10 μM, from about 5.0 μM to about 10 μM, from about 6.0 μM to about 10 μM, from about 7.0 μM to about 10 μM, from about 8.0 μM to about 10 μM, from about 9.0 μM to about 10 μM, about 0.5 μM, about 0.6 μM, about 0.7 μM, about 0.8 μM, about 0.9 μM, about 1.0 μM, about 2.0 μM, about 3.0 μM, about 4.0 μM, about 5.0 μM, about 6.0 μM, about 7.0 μM, about 8.0 μM, about 9.0 μM, or about 10 μM.

In some embodiments, the reversion media is serum-free, for example, free of animal serum (e.g., fetal bovine serum). Serum-free media may still contain undefined non-serum products such as serum albumin, growth factors, and hormones, among others. In some embodiments the reversion media is chemically-defined. Chemically-defined media requires that all of the ingredients of the media be identified and their exact concentrations known. Chemically-defined media does not contain fetal bovine serum, bovine serum albumin, or human serum albumin. In preferred embodiments, the reversion media does not contain any serum replacement, such as, for example, KnockOut™ Serum Replacement (Life Technologies, Carlsbad, Calif., USA), or equivalents thereof.

### Primed Media

As will be apparent to the skilled artisan upon reading this disclosure, the present disclosure provides cell culture media for culturing primed pluripotent stem cells. As used herein, the cell culture media for culturing a primed PSC may be referred to as “primed media.”

In some embodiments, the primed media comprises a basal media. The basal media can be any basal media known to those in the art. The basal media can be a mixture of different basal media. The basal media may be purchased from commercial sources. In some embodiments, the basal medium of the primed media comprises at least one of DMEM/F12-Glutamax, Neurobasal medium, N2 supplement (Life Technologies, Carlsbad, Calif., USA), B27 supplement (Life Technologies, Carlsbad, Calif., USA), BSA Fraction V (Life Technologies, Carlsbad, Calif., USA), and Glutamax (Life Technologies, Carlsbad, Calif., USA), or equivalents thereof. In some embodiments, the basal medium of the primed media comprises each of DMEM/F12-Glutamax, Neurobasal medium, N2 supplement (Life Technologies, Carlsbad, Calif., USA), B27 supplement (Life Technologies, Carlsbad, Calif., USA), BSA Fraction V (Life Technologies, Carlsbad, Calif., USA), and Glutamax (Life Technologies, Carlsbad, Calif., USA), or equivalents thereof.

In some embodiments, the DMEM/F12-Glutamax or equivalent thereof is present in the primed media at between about 25% and about 75% of the volume of the primed media. In preferred embodiments, the DMEM/F12-Glutamax or equivalent thereof is present in the primed media at about 50% of the volume of the primed media.

In some embodiments, the Neurobasal medium or equivalent thereof is present in the primed media at between about 25% and about 75% of the volume of the primed media. In preferred embodiments, the Neurobasal medium or equivalent thereof is present in the primed media at about 50% of the volume of the primed media.

In some embodiments, the N2 supplement or equivalent thereof is present in the primed media at between about 0.0005% and about 0.05% of the volume of the primed media. In preferred embodiments, the N2 supplement or equivalent thereof is present in the primed media at about 0.005% of the volume of the primed media.

In some embodiments, the B27 supplement or equivalent thereof is present in the primed media at between about 0.001% and about 0.1% of the volume of the primed media. In preferred embodiments, the B27 supplement or equivalent thereof is present in the primed media at about 0.01% of the volume of the primed media.

In some embodiments, the BSA Fraction V or equivalent thereof is present in the primed media at between about 0.00007% and about 0.007% (from a 7.5% solution) of the volume of the primed media. In preferred embodiments, the BSA Fraction V or equivalent thereof is present in the primed media at about 0.0007% (from a 7.5% solution) of the volume of the primed media.

In some embodiments, the Glutamax or equivalent thereof is present in the primed media at between about 0.00007% and about 0.007% of the volume of the primed media. In preferred embodiments, the BSA Fraction V or equivalent thereof is present in the primed media at about 0.0007% of the volume of the primed media.

In some embodiments, the primed media comprises a basal media and further comprises at least one additive. In some embodiments the at least one additive is, for example, basic fibroblast growth factor (bFGF), activin A or transforming growth factor-β (TGF-β). In some embodiments, the primed media comprises a basal media, bFGF and activin A. In some embodiments, the primed media comprises a basal media, bFGF and TGF-β. In some embodiments, the primed media comprises a basal media, bFGF, activin A and TGF-β.

In some embodiments, the primed media comprises a basal media and bFGF. The bFGF of the present disclosure can be at a concentration in the primed media, for example, from about 1 ng/mL to about 25 ng/mL, about 5 ng/mL to about 20 ng/mL, about 10 ng/mL to about 15 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, or about 25 ng/mL.

In some embodiments, the primed media comprises a basal media and Activin A. The Activin A of the present disclosure can be at a concentration in the primed media, for example, from about 5 ng/mL to about 50 ng/mL, about 10 ng/mL to about 40 ng/mL, about 15 ng/mL to about 30 ng/ml, about 15 ng/mL to about 25 ng/ml, about 5 ng/mL, about 10 ng/mL, about 15 ng/mL, about 20 ng/mL, about 25 ng/mL, about 30 ng/mL, about 35 ng/mL, about 40 ng/mL, or about 50 ng/mL.

In some embodiments, the primed media comprises a basal media and TGF-β. The TGF-β of the present disclosure can be at a concentration in the primed media, for example, from about 0.1 ng/mL to about 10 ng/mL, about 0.5 ng/mL to about 5 ng/mL, about 1 ng/mL to about 4 ng/mL, about 2 ng/mL to about 3 ng/mL, about 0.1 ng/mL, about 0.2 ng/mL, about 0.3 ng/mL, about 0.4 ng/mL, about 0.5 ng/mL, about 0.6 ng/mL, about 0.7 ng/mL, about 0.8 ng/mL, about 0.9 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, or about 10 ng/mL.

In some embodiments, the primed media comprises a basal media and beta-mercaptoethanol (2-ME). The 2-ME (1000×) of the present disclosure can be at a concentration in the primed media, for example, from about 0.0001% to about 0.01%. In preferred embodiments, the 2-ME is at a concentration in the primed media of about 0.001%.

In some embodiments, the primed media is serum-free, for example, free of animal serum (e.g., fetal bovine serum). Serum-free media may still contain undefined non-serum products such as serum albumin, growth factors, and hormones, among others. In some embodiments the primed media is chemically-defined. Chemically-defined media requires that all of the ingredients of the media be identified and their exact concentrations known. Chemically-defined media does not contain fetal bovine serum, bovine serum albumin, or human serum albumin. In preferred embodiments, the primed media does not contain any serum replacement, such as, for example, KnockOut™ Serum Replacement (Life Technologies, Carlsbad, Calif., USA) or equivalents thereof.

### Culture Conditions

The primed PSCs and naïve PSCs of the present disclosure can be cultured under any conditions known to those in the field. For example, any growth substrate may be used, for example, feeder cells (e.g., mouse embryonic feeders (MEFs) and mouse fibroblast STO cell transformed with murine LIF and neomycin resistance (SNL)), extracellular matrices (e.g., Matrigel®, Cultrex® BME PathClear, Geltrex®), gelatin, collagen, poly-lysine, poly-ornithine, fibronectin, vitronectin, or laminin, among others. In some embodiments, the cells are cultured on a layer of fibronectin. In some embodiments, the laminin is laminin-511, for example recombinant laminin-511) (iMatrix, Clontech, Mountain View, Calif., USA). In one preferred embodiment, the cells are cultured under feeder free conditions.

In some embodiments, the PSCs of the disclosure are cultured in conditions of 1-20% oxygen (O₂) and 5% carbon dioxide (CO₂). In some embodiments, the PSCs of the present disclosure are cultured under hypoxic conditions (e.g., in the presence of less than 10% O₂). In some embodiments, the PSCs of the present disclosure are cultured at about 37° C. In some embodiments, the PSCs of the present disclosure can be cultured at about 37° C., 5% CO₂ and 10-20% O₂.

In some embodiments, the naïve pluripotent stem cell and/or the primed pluripotent stem cell is cultured in hypoxic conditions for a period of time. For example, the naïve pluripotent stem cell and/or the primed pluripotent stem cell may be cultured under normoxic conditions (˜20% O₂) for a period of time and then switched to hypoxic conditions, for example ˜5% O₂. In other embodiments, the naïve pluripotent stem cell and/or the primed pluripotent stem cell may be cultured under normoxic conditions for a period of time and then switched to hypoxic conditions and culture in reversion media for a period of time. In other embodiments, the naïve pluripotent stem cell and/or the primed pluripotent stem cell may be cultured under normoxic conditions for a period of time and then switched to hypoxic conditions and cultured in reversion media for a period of time and then switched back to normoxic conditions in either the reversion media or conventional culture media. In yet other embodiments, the naïve pluripotent stem cell and/or the primed pluripotent stem cell may be cultured under hypoxic conditions in primed media for a period of time then cultured in reversion media while maintaining the hypoxic conditions.

### IV. Methods

As will be apparent to the skilled artisan upon reading this disclosure, the present disclosure provides methods of deriving naïve pluripotent stem cells. In one aspect this disclosure provides, methods for deriving a naïve pluripotent stem cell comprising: culturing a primed pluripotent stem cell in a culture media comprising an effective amount of an agonist of a lysophosphatidic acid receptor (LPAR), thereby reverting the primed pluripotent stem cell to a naïve pluripotent stem cell.

In one aspect, this disclose provides methods for maintaining a naïve pluripotent stem cell in its naïve state, comprising culturing the naïve pluripotent stem cell in a culture media comprising an effective amount of an agonist of a LPAR.

In one aspect, this disclosure provides methods for deriving naïve pluripotent stem cells comprising: culturing primed pluripotent stem cells in a culture media (i.e., a basal medium (e.g., StemFit)), comprising an effective amount of an agonist of an LPAR (e.g., OMPT and/or LPA), activin A, a MEK inhibitor, a GSK3β inhibitor, n-acetyl cysteine, and optionally bFGF.

In one aspect, this disclose provides methods for deriving naïve pluripotent stem cells comprising: culturing primed pluripotent stem cells in a culture media, wherein the culture media is substantially free of ERK1/2 inhibitor, GSK3β inhibitor, or both.

In some aspects, this disclose provides methods for deriving naïve PSCs, wherein primed PSCs differentiate into a primordial germ cell, followed by a stabilized naïve pluripotent state.

In some embodiments, the primed PSCs are cultured in the primed media for at least an hour, at least two hours, at least three hours, at least five hours, at least twelve hours, at least twenty-four hours, at least thirty-six hours, at least forty-eight hours. It is contemplated that the primed media acts, at least in part, to enhance heterochromatin formation. In particular, the primed media contains factors (e.g., bFGF, activin A, TGF-β) that act to condense chromatin, in the absence of factors that relax chromatin.

In some embodiments, the primed media is replaced by a naïve media. All, substantially all, or a portion of the primed media can be replaced with naïve media or a mixture of primed and naïve media can be added to the PSCs. For example, a media comprising a 75/25, 50/50, or 25/75 mixture of primed/naïve media can be added to the PSCs. PSCs can be cultured in a mixed primed/naïve media for any amount of time, for example, at least an hour, at least two hours, at least three hours, at least five hours, at least twelve hours, at least twenty-four hours, at least thirty-six hours, at least forty-eight hours. It is contemplated that use of a mixed primed/naïve media will slow the reversion rate by acting, at least in part, to slow the relaxation of the PSC chromatin.

In some embodiments, the primed PSCs are cultured in the naïve media for at least an hour, at least two hours, at least three hours, at least five hours, at least twelve hours, at least twenty-four hours, at least thirty-six hours, at least forty-eight hours or longer. It is contemplated that the naïve media acts, at least in part, to relax heterochromatin. In particular, the naïve media contains factors (e.g., an LPAR agonist, a BMP) that act to relax chromatin, and in some embodiments, in the absence of factors that condense chromatin (e.g., bFGF, activin A, TGF-β).

In some embodiments, PSCs are seeded (e.g., 20,000 cells/well of a 6-well plate) in primed media. Approximately twenty-four hours after the cell seeding, the primed media is removed and replaced with reversion media. Reversion media is replaced every twenty-four hours.

In some embodiments, the primed PSC reverts to the naïve PSC in about twenty days or less, about fifteen days or less, about ten days or less, about five days or less, or about three days or less when cultured in either a mixed primed/naïve media or a naïve media. In some embodiments, the primed pluripotent stem cell is cultured for an effective amount of time that does not exceed 40 days, 30 days, 20 days, 15 days, 10 days, 5 days, or 3 days. In preferred embodiments, the amount of time does not exceed 10 days.

In some embodiments, a naïve PSC can be cultured in either primed media or a conventional PSC media and remain a naïve cell.

In some embodiments, the primed pluripotent stem cell reverts to the naïve pluripotent stem cell in about twenty days or less, about fifteen days or less, about ten days or less, about five days or less, or about three days or less.

In some embodiments, greater than about 5%, greater than about 10%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, greater than about 97%, greater than about 99%, or 100% of the primed PSCs revert to naïve PSCs.

In some embodiments, the naïve PSC maintain a normal karyotype for more than about 5 passages, about 10 passages, about 15 passages, about 20 passages, about 30 passages, about 40 passages, about 50 passages, about 60 passages, about 70 passages, or about 80 passages.

In some embodiments, the naïve PSC is capable of being maintained in the naïve, undifferentiated and pluripotent state with a normal karyotype for more than about 5 passages, about 10 passages, about 15 passages, about 20 passages, about 30 passages, about 40 passages, about 50 passages, about 60 passages, about 70 passages, about 80 passages.

Many methods exist for identifying whether a primed cell has reverted to a naïve cell. Non-limiting examples include: expression levels of pluripotency makers (e.g., Oct4, Sox2, Nanog, Klf2, Klf4); expression of naïve state markers (e.g., PRDM14, CD31 (PECAM1)); X chromosome inactivation status (XCI) markers (e.g., Artx, Tsix); ability to incorporate into chimeras; response to Lif/Stat3⁺ culture conditions; and response to Fgf/Erk⁺ culture conditions, among others. Expression levels of pluripotency markers, naïve state makers, etc. can be detected by any method known to those in the art. For example, fluorescence activated cell sorting (FACS) or flow cytometry can be used to detect levels membrane-bound and cell surface markers, for example, CD31 expression. Immunohistochemistry/immunocytochemistry can be employed for detecting levels of extracellular and intracellular markers, for example, pluripotency markers, naïve state markers, and XCI status markers. In addition, transcript expression of pluripotency markers and naïve state markers can also be detected by methods such as quantitative real-time polymerase chain reaction (qPCR) or real time-qPCR (RT-qPCR).

The methods of this disclosure also contemplate further monitoring the PSCs for their differentiation which can be determined upon visual examination of the cell morphology and/or examination of cell- or tissue-specific markers indicative of differentiation. For example, glial fibrillary acid protein (GFAP) and sex determining region Y-box 1 (Sox1) are common ectoderm markers. Brachyury and Goosecoid are common early mesoderm lineage markers, while Sox17 and pancreatic and duodenal homeobox 1 (Pdx1) are common endoderm markers. In addition, the degree of cell differentiation within a population of stem cells can be determined by expression levels of stage-specific embryonic antigens (SSEA) (SSEA-1 or SSEA-4), Tra-1-60, Tra-1-81. Undifferentiated PSCs express high levels of SSEA4, Tra-1-60 and Tra-1-81 and expression is rapidly downregulated upon differentiation. Another marker commonly used for assessing differentiation is alkaline phosphatase.

### V. Reprogramming

The methods of this disclosure also contemplate using reversion media, primed media, or both to improve reprogramming efficiencies of somatic cells (e.g., fibroblasts) into iPSCs. In some embodiments, it is contemplated that using reversion media, primed media, or both will allow fewer factors of conventional methods to generate iPSCs. Methods of reprogramming are well-known in the art and include, for example, Takahashi et al., (2007) Cell 131(5):861-872; Yu et al., (2007) Science 318(5858):1917-1920; Shi et al., (2008) Cell Stem Cell 2(6):525-528; Nakagawa et al., (2008) Nat. Biotechnol. 26(1):101-106; Okita et al. (2013) Stem Cells 31(3):458-466.

In some embodiments, the reprogramming methods can be performed in primed media. In other embodiments, the reprogramming methods can be performed in naïve media. In yet other embodiments, the reprogramming methods can be performed in normal fibroblast medium (10% FBS in DMEM). In some embodiments, following introduction of reprogramming factors, the cells (e.g., fibroblasts) are transferred and cultured in naïve media. In some embodiments, there is an increase in reprogramming efficiency as compared to conventional reprogramming methods of about 5-fold, about 10-fold, about 20-fold, about 30-fold, about 40-fold, about 50-fold, about 60-fold, about 70-fold, about 80-fold, about 90-fold, about 100-fold, or more. In some embodiments, the iPSCs derived in naïve media exhibit a more undifferentiated phenotype, for example, express high levels of the pluripotency factors Oct4, Nanog, Sox2, Klf2 and Klf4; self-renew in response to either Lif/Stat3 or 2i (ERKi/GSKi); differentiate in response to Fgf/Erk; and/or exhibit a XaXa X-chromosome status. It is contemplated that reprogramming efficiencies are higher when iPSCs are derived in naïve media as compared to conventional culture media because the chromatin is in a more relaxed state and factors such as, activin A, bFGF, or both are absent.

In some aspects, provided herein are compositions of comprising a population of isolated naïve pluripotent stem cells prepared by any one of the methods of using any of the media disclosed and described above. In some embodiments, the composition comprise naïve pluripotent stem cells and a pharmaceutical acceptable excipient.

The composition can comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions may be prepared using conventional techniques that may include sterile isotonic saline, water, 1,3-butanediol, ethanol, 1,2-propylene glycol, polyglycols mixed with water, Ringer's solution, etc.

Compositions may include a preservative and/or a stabilizer. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorobutanol, 2-ethoxyethanol, and imidurea.

In some embodiments, the composition may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

### VI. Kits

Also disclosed are kits comprising: (a) a cell culture media for reverting a primed pluripotent stem cell into a naïve pluripotent stem cell, the media comprising an agonist of a LPAR; and (b) instructions. In some embodiments, the kits further comprise (b) bFGF, activin A, transforming growth factor-β (TGF-β), or any combination thereof. In some embodiments, the kits further comprise isolated naïve pluripotent stem cells. In other embodiments, the kits further comprise isolated primed pluripotent stem cells.

The components of the kit may be contained in one or different containers such as one or more vials. The cell culture media may be in liquid or solid form (e.g. after lyophilization) to enhance shelf-life. If in liquid form, the components may comprise additives that enhance shelf-life.

In various embodiments, instructions for use of the kits will include directions to use the kit components for deriving a naïve pluripotent stem cell or for maintaining a naïve pluripotent stem cell in its naïve state. The instructions may further contain information regarding how to prepare (e.g., dilute, in the case of concentrated media) the media and the pluripotent stem cells (e.g., thawing and/or culturing).

### EXAMPLES

The following examples are intended to further illustrate certain embodiments of the disclosure. The examples are put forth so as to provide one of ordinary skill in the art and are not intended to limit its scope.

Mouse embryonic stem cells (ESCs) and induced pluripotent stem cells (miPSCs) represent naïve pluripotency, while mouse epiblast stem cells (mEpiSCs) and most human ESCs and iPSC lines represent primed pluripotency. Primed and naïve cells differ in their cytokine requirements. Consistent with different signaling pathways functioning in these two classes of cells, naïve and primed cells exhibit unique gene expression patterns and epigenetic features. One epigenetic feature that differs between primed and naïve stem cell is X chromosome inactivation (XCI), the mammalian dosage compensation mechanism that equalizes X-linked gene dosage between XX females and XY males. In vitro, naïve mouse female cells have two active X chromosomes (Xas), reflecting the Xa/Xa status of the naïve cells of the female inner cell mass. In contrast, primed cells have one Xa and one inactive X (Xi), showing the transcriptional silencing of one X chromosome that is seen in the cells of the post-implantation embryonic ectoderm, from which EpiSCs are derived.

Culture conditions, including cytokines, extracellular matrix components, and small molecules, can impact epigenetic features in pluripotent stem cells. For example, mouse naïve cells are maintained in LIF containing medium and can efficiently be converted into primed cells, by transfer into Activin A and bFGF containing medium. This transition is accompanied in by genome-wide alterations in DNA methylation patterns.

In contrast, conversion of primed cells to naïve cells is inefficient. mEpiSCs are converted to mESC-like cells after several passages on mouse embryo fibroblast (MEF) feeders in LIF containing medium. This LIF-dependent conversion can be boosted and accelerated by overexpression of Nanog, KLF2, KLF4, and/or Prdm14, although the conversion efficiencies are low. The difficulty in converting primed cells to naïve cells may be due to epigenetic barriers that inhibit the transition and/or may indicate that the appropriate signaling molecules remain to be elucidated. Because of their extensive differentiation capacity, naïve pluripotent stem cells are an important source of material for regenerative medicine. Many established methods for generating naïve cells involve genetic manipulations or using chemical inhibitors to promote conversion of less plastic primed stem cells to naïve pluripotency. However, these non-physiological manipulations may not be suitable for regenerative medicine.

It was previously reported that culture conditions impact XCI status in human iPSCs (hiPSCs) (Tomoda et al. (2012) Cell Stem Cell 11:91-99). Human iPSC lines reprogrammed and maintained on LIF expressing SNL feeder cells (McMahon et al., (1990) Cell 62:1073-1085) are predominantly XaXa, while hiPSC lines derived on MEF feeder cells are mainly XaXi. Early passage XaXi hiPSCs are converted to XaXa after several divisions on SNL feeder cells. It remains to be determined whether the SNL feeder-cell culture condition contains factors, including LIF, that promote conversion from primed to naïve states.

### Example 1. Culture Conditions that Affect the X Chromosome Inactivation Status in mEpiSCs

During somatic cell reprogramming the human Xi is reliably reactivated in hiPSCs reprogrammed on SNL feeder cells. This result suggests that the SNL feeder culture condition contains factors that induce the Xi-reactivation. It was first determined whether the culture conditions that promoted reactivation of the Xi during reprogramming also promoted Xi-reactivation of mouse EpiSCs, which are XaXi. Results of culturing an X-GFP mEpiSC reporter line, in which the green fluorescent protein (GFP) gene is located on the X-chromosome in different media were assayed by fluorescence microscopy. Gillich et al., (2012) Cell Stem Cell 10(4):525-439, Bao et al., (2009) Nature 461(7268):1292-1295. Xi-GFP mEpiSCs were kindly provided by Drs. Azim Surani and Siqin Bao and were routinely maintained on a fibronectin (Sigma, St. Louis, Mo., USA)-coated plate (feeder free condition) in N2B27 basal medium (Ndiff 227 medium from StemCells Inc (Palo Alto, Calif., USA) and later from Clontech (Mountain View, Calif., USA) supplemented with 20 ng/ml Activin A (R&D systems (Minneapolis, Minn., USA)) and 12 ng/ml bFGF (Millipore (Billerica, Mass., USA)). Cells were passaged every two to three days at a 1:20 dilution from a previous culture after detached/scraped and dissociated with Accutase (Millipore (Billerica, Mass., USA)).

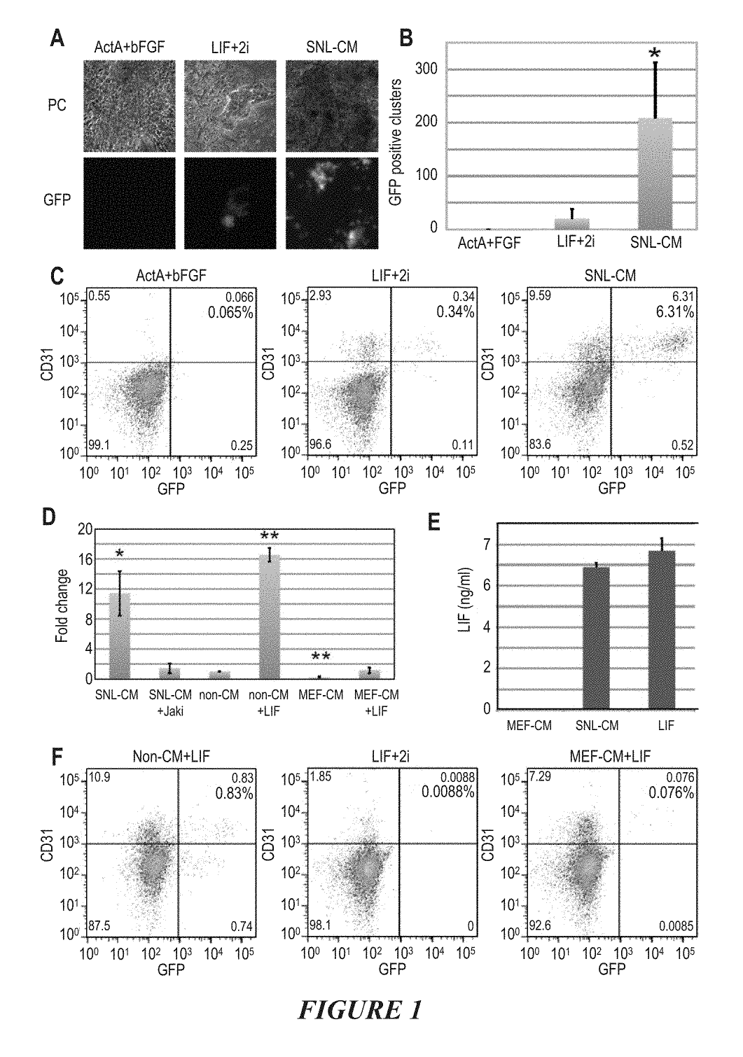

Cells remain GFP negative when cultured under feeder-free mEpiSC conditions, N2B27 medium containing Activin A and bFGF on fibronectin coated plates (FIG. 1, panels A and B). A fraction of the cells become GFP positive when shifted to feeder-free mESC media, N2B27 containing LIF and two chemical inhibitors for MAPK and GSK3b (LIF+2i) on fibronectin coated plates (FIG. 1, panels A and B), paralleling the behavior of these cells cultured in other mESC culture conditions (Bao et al., (2009) Nature 461(7268):1292-1295, Greber et al., (2010) Cell Stem Cell 6(3):215-226). X-GFP mEpiSCs cultured in human ESC medium, Knockout DMEM supplemented with Knockout Serum Replacement (KSR), conditioned with SNL feeders (SNL-CM) on fibronectin coated plates exhibited a dramatic increase in the number of GFP positive cells (FIG. 1, panels A and B).

To more quantitatively assess the proportion of GFP positive cells under these three culture conditions, Fluorescence Activated Cell Sorting (FACS) was employed. For flow cytometric analyses, Xi-GFP mEpiSCs were dissociated into single cell suspensions with Accutase. The single cells were stained with a CD31 (PECAM) antibody (Clone MEC13.3/BD Bioscience (Franklin Lakes, N.J., USA)) conjugated with allophycocyanin, or APC. The stained cells were analyzed using MACSQuant VYB (Miltenyi Biotec, San Diego, Calif., USA) and FlowJo (FlowJo LLC, Ashland, Oreg., USA) software. The flow cytometric analyses revealed that more than 5% cells become GFP⁺ in the SNL-CM, while less than 1% cells become GFP⁺ in the LIF+2i medium (FIG. 1, panel C). In the LIF+2i and SNL-CM, the GFP⁺ populations were also CD31⁺, a marker for mouse naïve pluripotent stem cells (FIG. 1, panel C). Thus, these results suggest that the SNL-CM not only reactivates the Xi in the X-GFP mEpiSCs but also reverts the primed cells into naïve pluripotent stem cell-like cells more efficiently than the LIF+2i medium.

During human reprogramming, LIF contributed to Xi-reactivation (Tomoda et al. (2012) Cell Stem Cell 11:91-99). Therefore, it was investigated whether LIF signaling plays a role in Xi-reactivation in Xi-GFP mEpiSCs. First, the effects of a JAK kinase inhibitor that inhibits the LIF-JAK-STAT3 signaling pathway were examined. Addition of the JAK kinase inhibitor significantly reduced the number of GFP⁺ clusters that emerged in SNL-CM (FIG. 1, panel D). Next, the effect of employing conditioned medium that does not contain LIF, by culturing X-GFP mEpiSCs in human ESC medium without conditioning (non-CM) was examined. Few GFP⁺ cell clusters emerged in the non-CM (FIG. 1, panel D). Addition of LIF in an amount comparable to that in SNL-CM (FIG. 1, panel E) to the non-CM significantly enhanced the production of GFP+ clusters in the culture (FIG. 1, panel D). Together these results indicate that LIF promotes reactivation of the Xi in mEpiSCs.

It was also examined whether LIF could stimulate Xi-reactivation in medium conditioned by mouse embryo fibroblasts (MEF-CM), which do not express LIF (FIG. 1, panel E). In contrast to non-CM+LIF, MEF-CM+LIF did not support efficient Xi-reactivation (FIG. 1, panel D). The flow cytometric analyses also showed that adding LIF to MEF-CM does not induce GFP expression to the same extent as non-CM, although CD31 expression is induced in the MEF-CM+LIF (FIG. 1, panel F). These results suggest that the expression of CD31 is not linked to X-inactivation status, and that MEF-CM contains factors that counteract LIF function in the Xi-reactivation.

### Example 2. Activin A and bFGF Suppress Xi-Reactivation

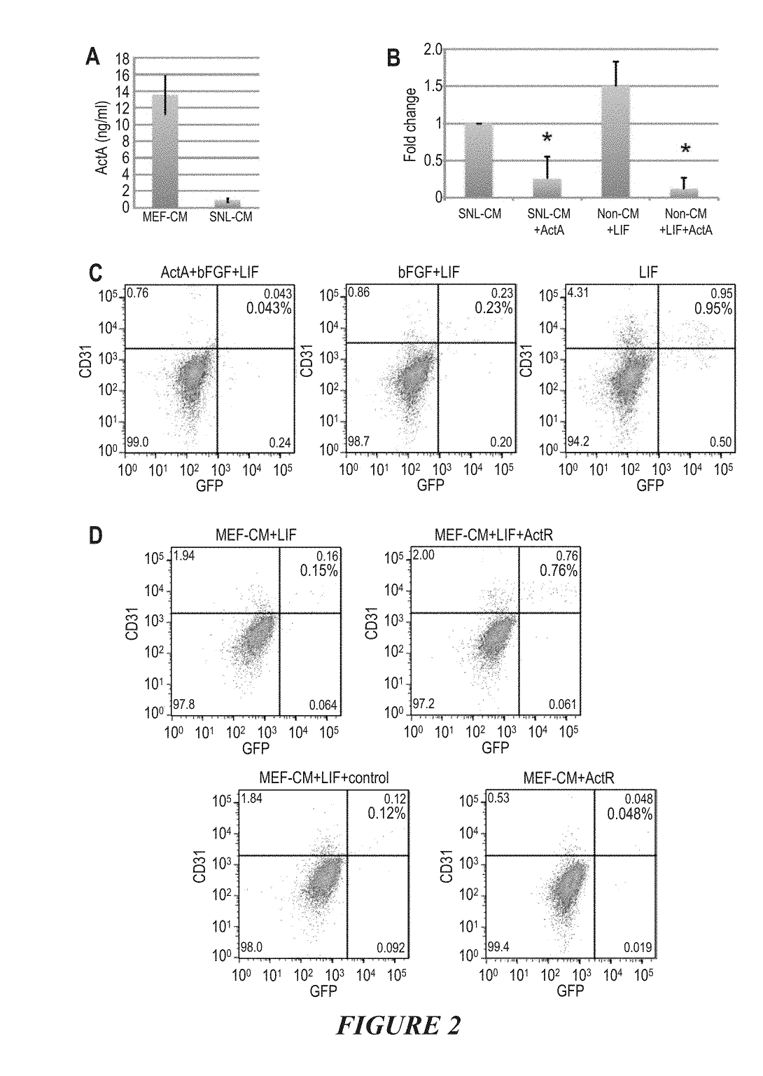

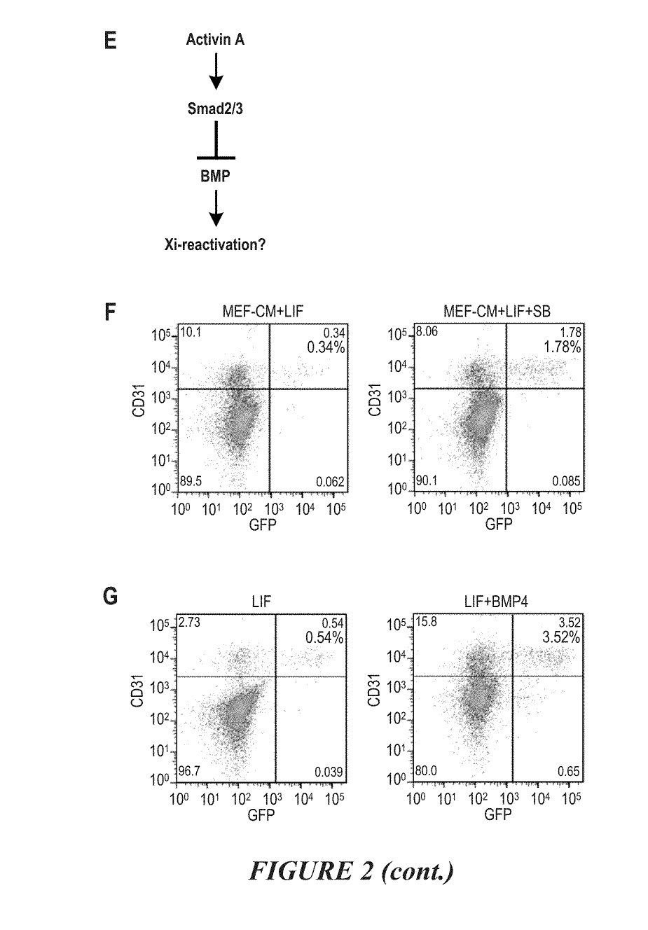

Activin A and bFGF are required for maintaining primed pluripotency in mEpiSCs, suggesting that they may be candidates for the MEF-CM activity that antagonizes LIF-mediated Xi-reactivation. All the non-CM media used in the previous experiments were supplemented with bFGF, therefore the initial focus was on a potential role for Activin A. The concentrations of LIF and Activin A in the indicated media were determined using ELISA. Mouse LIF was used and human/mouse/rat Activin A Quantikine ELISA kits (R&D Systems (Minneapolis, Minn., USA)), according to the manufacturer's instructions. It was found that MEF-CM contains Activin A at 11 ng/ml while SNL-CM contains Activin A only at 1 ng/ml (FIG. 2, panel A). To examine whether this factor impacts Xi-reactivation, the effects of adding exogenous Activin A on LIF-mediated Xi-reactivation, using SNL-CM or non-CM containing LIF were assessed. It was found that addition of Activin A significantly reduced numbers of GFP+ clusters both in the SNL-CM and non-CM+LIF (FIG. 2, panel B).

It was next asked whether Activin A could inhibit LIF-mediated Xi-reactivation in mEpiSCs using FACS analyses. When LIF was added to mEpiSC medium, which contains bFGF and Activin A, there is no significant GFP/CD31 double positive cells (FIG. 2, panel C). When Activin A was removed from the LIF-containing media, an increase in GFP/CD31 double positive cells was observed. Removing Activin A and bFGF caused a further increase. These results suggest that both Activin A and bFGF have negative effects on the LIF induced Xi-reactivation.

To investigate whether Activin A counteracts the LIF effect in the MEF-CM, Activin A signaling was inhibited with the Activin A receptor complex (ActR complex), which sequesters Activin A and suppresses downstream signaling pathways. While adding LIF in MEF-CM did not induce GFP/CD31 double positive cells efficiently, addition of the ActR complex, but not a control protein, in the medium increased the % of the double positive cells (FIG. 2, panel D). Adding the ActR complex without LIF did not induce GFP (FIG. 2, panel D), indicating that the ActR effect is LIF-dependent. Thus, Activin A or its family members attenuate the LIF effect on Xi-reactivation in MEF-CM.

The SMAD2/3 transcription factors lie downstream of Activin A, and mediate several of this cytokine's biological effects (FIG. 2, panel E). To construct shRNA expression vectors, target sequences were cloned into a piggyBac vector that was generated from cloning a U6-driven BsmbI cut site. The original lentiviral construct was kindly provided by Dr. Mohammad Mandegar from which the U6-BsmbI-CNKB region was cloned. shRNA were designed as two complementary oligonucleotides against targets, containing a polyT 3′ to the shRNA sequence and unique directionally specific sticky ends to the BsmbI cut sites. For each, the two complementary oligos were annealed and then ligated into BsmbI digested piggyback vector and sequenced for verification. The shRNA expression vectors were transfected into Xi-GFP mEpiSCs using Lipofectamine 2000. For the transfection, cells were seeded on a fibronectin-coated plate with a DNA (the shRNA expression vector together with piggyBac transposase expression vector) and Lipofectamine mixture in a medium containing Activin A and bFGF. Twenty-four hours after the transfection, we replenished the flesh medium without the DNA-Lipofectamine mixture and then change the medium every 24 hours. Seventy-two hours after the transfection, selection for shRNA expressing cells was performed using 10 μg/ml Blasticidin S (Life Technologies (Carlsbad, Calif., USA) in the medium. After four to five day selection, the conversion was started with the selected cell population. At the end of the conversion, the populations which highly expressed mKate fluorescent proteins were sorted by FACS Aria (BD Bioscience (Franklin Lakes, N.J., CA, USA)) to analyze the % of GFP⁺ cells and to extract RNA for the gene expression analyses.

To examine if SMAD2/3 plays a role in the Xi-reactivation SB431542, a chemical inhibitor of SMAD2/3 activation, was utilized. It was found that adding SB431542 enhances induction of the double positive cells in MEF-CM containing LIF (FIG. 2, panel F). Knockdown of SMAD2 by shRNA had a similar effect as treating cells with the inhibitor (data not shown). These results suggest that Activin A exerts its negative effect on LIF-induced Xi-reactivation through SMAD2/3 transcription factors.

It was recently reported that knocking down SMAD2 in mEpiSCs and human pluripotent stem cells activates BMP signaling pathways (FIG. 2, panel E) (Sakaki-Yumoto et al., (2013) J. Biol. Chem. 288(25):18546-18560). Thus, it was next examined if BMP enhances Xi-reactivation. Adding BMP4 alone in a N2B27 basal medium (EpiSC medium without bFGF or Activin A) did not induce GFP/CD31 double positive cells (data not shown). However, adding BMP4 in the N2B27 basal medium containing LIF dramatically increased the % of the double positive cells compared with LIF alone (FIG. 2, panel G). These results suggest that the Activin A-SMAD2/3 axis suppresses the LIF-induced Xi-reactivation and one possible mechanism of the suppression would be by repressing BMP signaling pathways.

### Example 3. Ascorbic Acid and LPA Enhance Xi-Reactivation

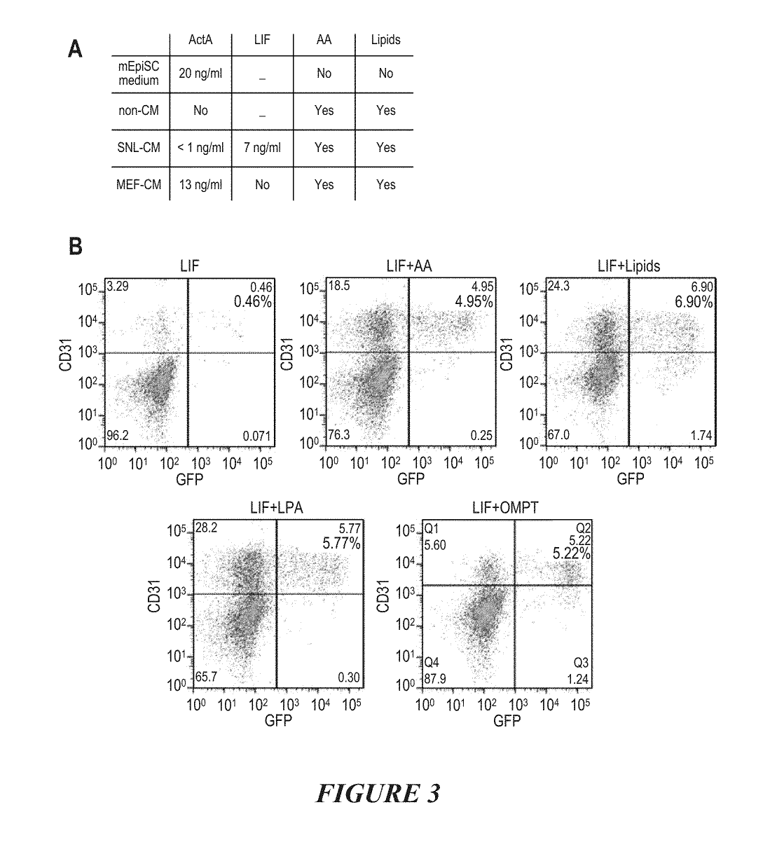

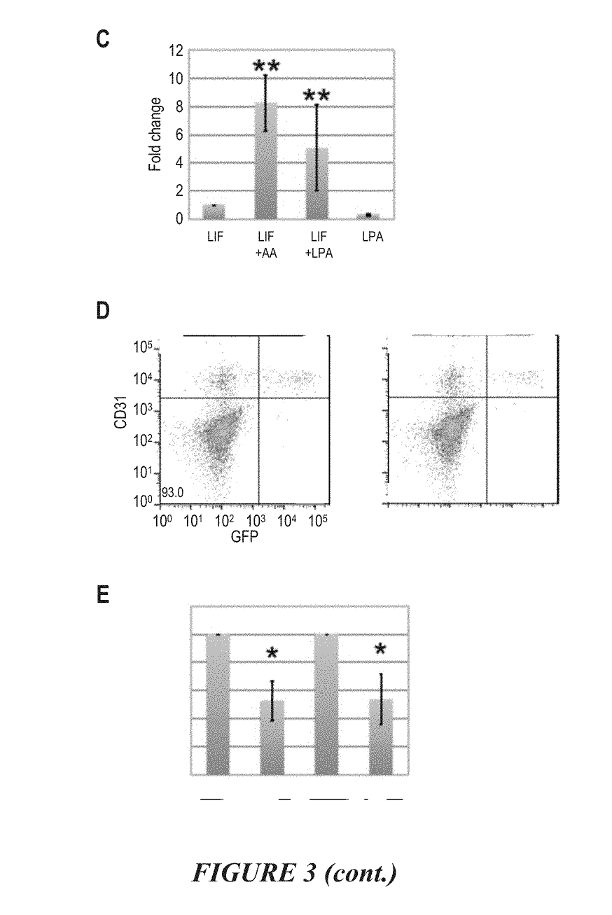

In contrast to media that did not efficiently induce Xi-reactivation, the two media that produced the greatest Xi-reactivation, SNL-CM and non-CM+LIF, both contain ascorbic acid and lipid mixture from KSR (Garcia-Gonzalo et al., PLoS One 3(1):e1384) (FIG. 3, panel A). Thus, it was examined whether ascorbic acid and the lipid mixture have ability to promote Xi-reactivation, by supplementing N2B27 medium with these small molecules. It was found that addition not only of ascorbic acid but also of the lipid mixture increased the % of GFP/CD31 double positive cells compared with LIF alone (FIG. 3, panels B and C). Thus, both components have positive effects on LIF-induced Xi-reactivation.

Mass spectrometry was used to assess the lipid component of KSR. Lysophosphatidic acid (LPA) was identified in low amounts (approximately 10 nM) in some batches of KSR (data not shown). Therefore, it was examined whether LPA affects Xi-reactivation. Addition of LIF and the KSR lipid mixture to N2B27 media resulted in a significantly greater increase in the proportion of GFP⁺CD31⁺ cells than only LIF. Using LPA or OMPT, a synthetic LPA (Hasegawa et al. (2003) Biol. Chem. 278:11962-11969), as the sole source of lipid caused a similar increase in the % of GFP⁺CD31⁺ cells, suggesting that LPA is a major contributor to the lipid mixture's activity (FIG. 3, panels B and C). Addition of LPA without LIF also induced GFP⁺ cells, albeit less efficiently than with LIF (FIG. 3, panel C). Thus, it was concluded that LPA and LIF are sufficient to efficiently induce Xi-reactivation.

LPA signals through a family of six LPA receptors (LPAR), and it was determined that LPAR1-3 were highly expressed in mEpiSCs (data not shown). OMPT signals predominantly through LPAR2 and LPAR3, suggesting that these LPARs may be sufficient for LPA to mediate Xi-reactivation. Ki16425, a LPAR1/3 competitive chemical inhibitor, was used to determine the contribution of LPAR1 and LPAR3. It was found that addition of Ki16425 significantly reduces % of GFP⁺ cells in medium containing LIF+LPA or LIF alone (FIG. 3, panel E). Thus, it was concluded that LPA affects Xi-reactivation partly through LPAR1/3 and endogenous LPA-LPAR1/3 signaling is involved in the LIF-induced Xi-reactivation.

Lipid signaling pathways are emerging as important players in maintaining pluripotency (Blaukwamp et al., (2012) Nat. Comm. 3(1070):1-10, Garcia-Gonzalo et al., PLoS One 3(1):e1384, Lian et al. (2010) Genes Dev. 24:1106-1118, Ohgushi et al. (2015) Cell Stem Cell 17:448-461). Surprisingly, the data here suggests that these signaling pathways are also important for converting primed to naïve pluripotent cells. Addition of lipids, LPA or OMPT, promoted conversion. However endogenous lipids may also play a role because the competitive inhibitor for LPAR1/3 impacted conversion in the medium that does not contain exogenous LPA. This result suggests unanticipated cross talk between the lipid signaling and LIF pathways. Inhibitor and knockdown studies indicate that LPAR1 plays a significant role in conversion, possibly by reducing NANOG expression in addition to affecting the LIF signaling activity.

OMPT is a specific agonist for LPAR2/3 (Hasegawa et al. (2003) Biol. Chem. 278:11962-11969). However, interestingly here it was found that knockdown of LPAR1 and not LPAR3 inhibited conversion with LBAO. Additionally, while omitting OMPT from LBAO medium resulted in a significant decrease in efficiency of conversion, this decrease was smaller than seen when each of the other factors was omitted. Thus, it may be that OMPT promotes the conversion by weak signaling through LPAR1, which predicts that a specific agonist for LPAR1, which is not reported so far, would further enhance conversion. Thus, developing specific agonists and antagonists for LPARs would be beneficial for controlling pluripotency ex vivo and would contribute to regenerative medicine.

### Example 4. The Combination of LIF, BMP4, Ascorbic Acid, and OMPT (LBAO) Efficiently Reactivate Xi and Convert Mouse Primed PSCs to Naïve PSCs

Xi-GFP mEpiSCs were dissociated with Accutase into single cell suspensions, and the single cells were seeded on a fibronectin-coated plate in the Activin A and bFGF-containing medium with two different cell densities (20,000 cells or 3,000 cells/well of a 6-well plate) one day before starting conversion experiments. Twenty-four hours after the seeding, fresh primed media was replenished as a negative control or changed to naïve medium. Assay medium was changed every 24 hours and GFP fluorescent was checked daily by microscopic inspection for eight (with 20,000 starting cells) to 13 days (with 3,000 starting cells).

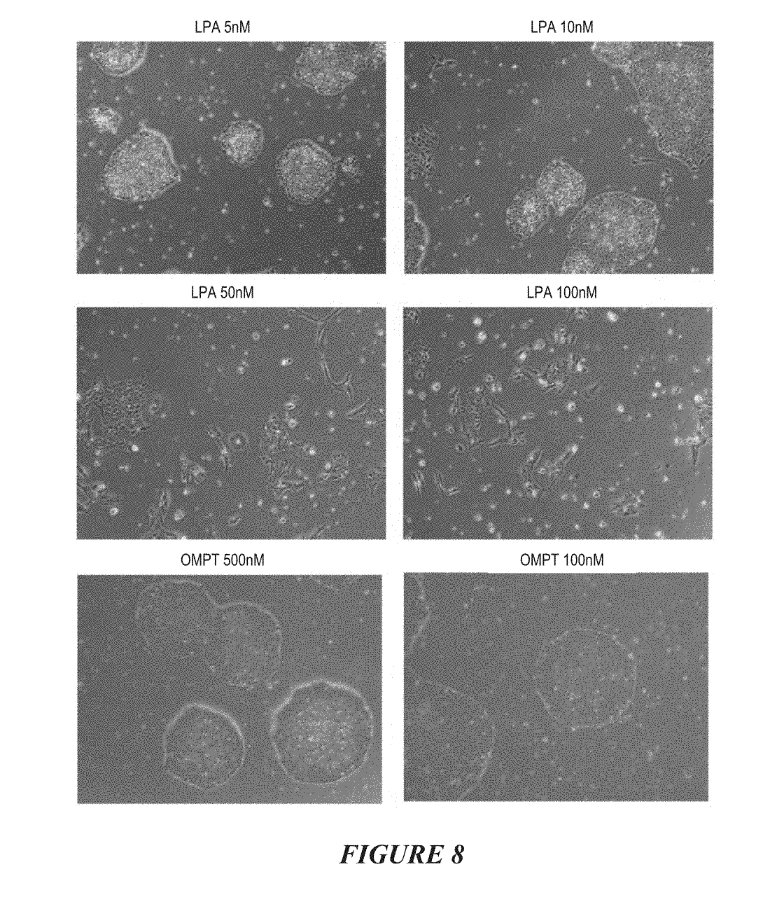

To optimize the concentration of LPA and OMPT, primed pluripotent stem cells were grown in various concentrations of LPA or OMPT. As shown in FIG. 8, at high concentrations (50 nM and 100 nM), LPA was toxic to primed pluripotent stem cells. On the other hand, primed pluripotent stem cells grown in media with OMPT concentrations of 100 nM and 500 nM appeared healthy and maintained colony morphologies characteristic of pluripotent stem cells.

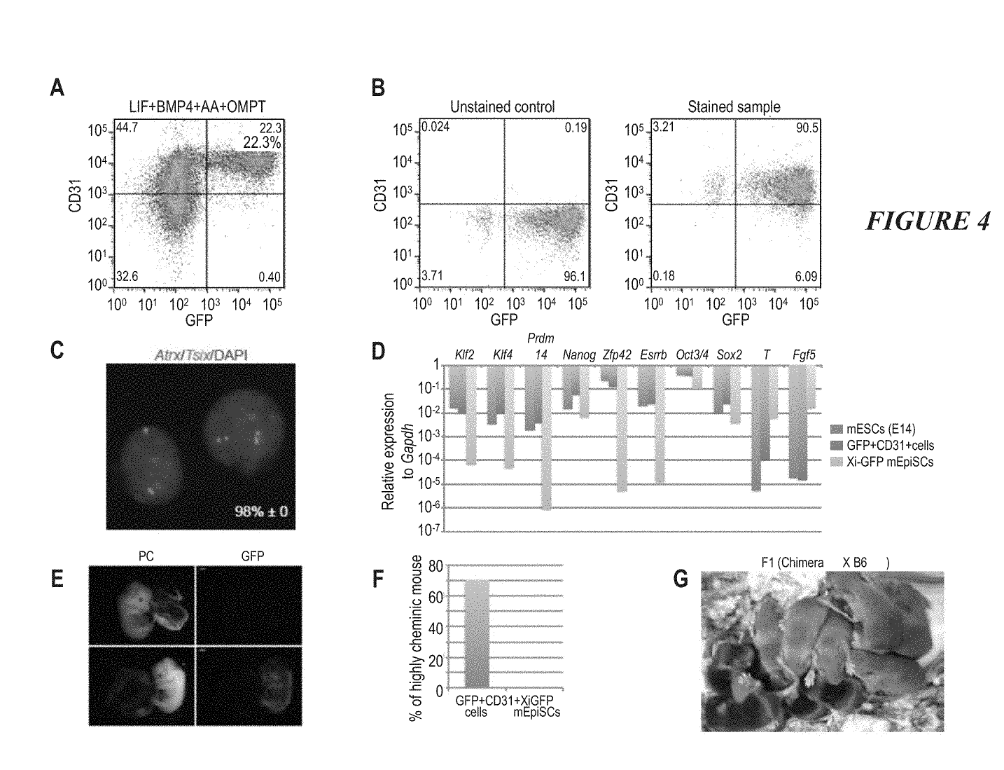

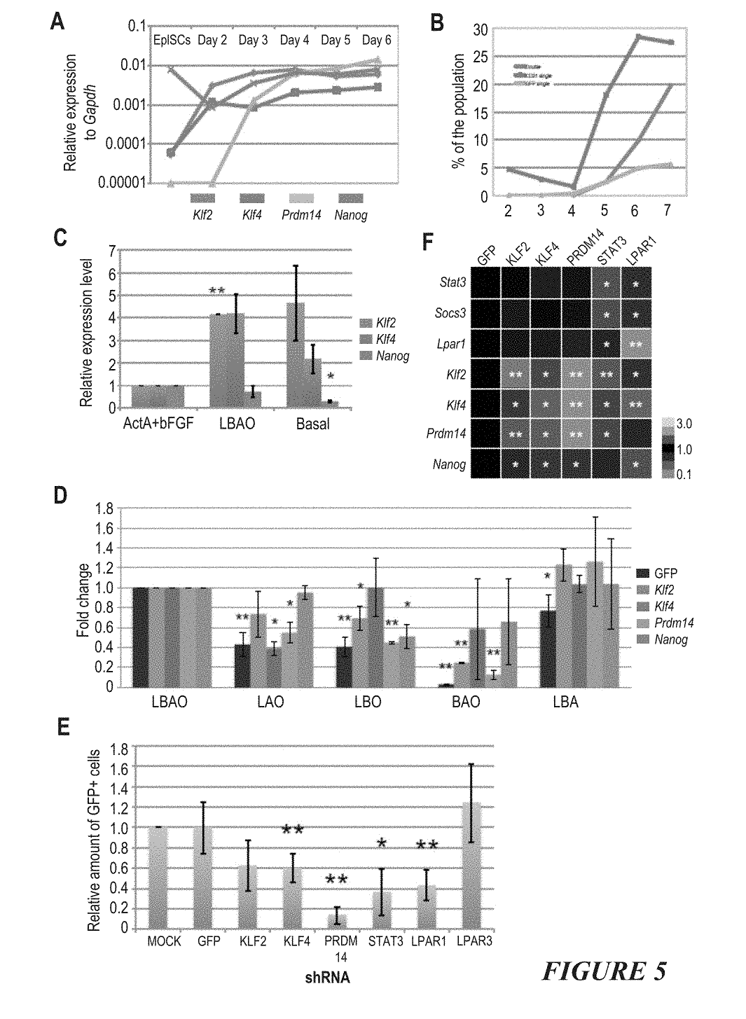

The naïve media contained 1,000 units LIF (Millipore), 10 ng/ml BMP4 (R&D systems), 64 μg/ml L-ascorbic acid 2-phosphate (Sigma), 10 nM to 100 nM LPA (Avanti Polar Lipids (Alabaster, Ala., USA)), and/or 100 nM OMPT (Avanti Polar Lipids (Alabaster, Ala., USA)) in N2B27 basal medium purchased from StemCells Inc (Palo Alto, Calif., USA) or prepared in house for use in FIGS. 4 and 5. The in house N2B27 medium consists of 500 ml DMEM/F12+GlutaMax (Life Technologies (Carlsbad, Calif., USA), 500 ml Neurobasal medium (Life Technologies (Carlsbad, Calif., USA)), 5 ml N2 supplement (Life Technologies (Carlsbad, Calif., USA)), 10 ml B27 supplement (Life Technologies (Carlsbad, Calif., USA)), 666 μl 7.5% BSA Fraction V (Life Technologies (Carlsbad, Calif., USA)) and 5 ml GlutaMax (Life Technologies (Carlsbad, Calif., USA)/1 liter. At the end of the conversion experiments, the conversion efficiencies were evaluated by counting GFP⁺ cell clusters or by flow cytometer.

To select GFP⁺CD31⁺ cells, cells cultured in LBAO medium for six to nine days were passaged and cultured on a laminin 511, or iMatrix, (Nippi/Iwai North America)-coated plate in 50:50 of LBAO/LIF+2i medium. Next day, the medium was changed to LIF+2i medium. Within a few passages under the culture condition, almost pure GFP⁺CD31⁺ cell population were yielded.

Based on the findings described above, it was examined if combining LIF, BMP4, ascorbic acid and LPA (or OMPT), in the absence of Activin A and bFGF, strongly reactivates the Xi. It was found that addition of each of LIF, BMP4, ascorbic acid and LPA (or OMPT) in a N2B27 medium efficiently induced GFP⁺CD31⁺ cells (FIG. 4, panel A). On average, 17.7±5.9% cells became the double positive cells at day 8. Additionally, more than 32.1±8% and 2.8±2.1% cells became CD31 and GFP single positive cells, respectively.

It was next examined if the GFP⁺CD31⁺ cells can be established as a cell line. Indeed, reverted cell culture was expanded well in LIF+2i medium, and GFP⁺CD31⁺ cell lines were easily established without picking up colonies (FIG. 4, panel B). Probe preparation and FISH procedure were described. Briefly, the FISH probes were prepared with Bioprime kit (Life Technologies (Carlsbad, Calif., USA), according to the manufacturer's instruction. GFP⁺CD31⁺ cells were harvested using Accutase to make a single-cell suspension and then the single cells were cytospun on a glass slide with Cyotospin (Thermo Scientific (Waltham, Mass., USA)) at 800 rpm for 3 min. The cytospun samples were permeabilized, fixed with 4% PFA for 10 minutes and stored in 70% EtOH at 4 degrees at least for overnight. The samples were then dehydrated in 85-100% EtOH and hybridized with the probes at 37 degrees overnight. After the hybridization, the samples were extensively washed and then mounted with antifade mounting medium (Vectashield, Vectorlabs (Burlingame, Calif., USA)) on a slide glass. The stained samples were microscopically examined with 100× oil lens. More than 200 cells in one sample were scored. Fluorescent In Situ hybridization with two X-linked gene, atrx and tsix, probes revealed that almost all GFP⁺CD31⁺ cells exhibit two nascent transcript foci for both atrx and tsix in a nucleus, suggesting they are XaXa (FIG. 4, panel C).

Expression of pluripotent marker and differentiation genes were tested. Total RNA was purified with Trizol reagent (Life Technologies (Carlsbad, Calif., USA)) and treated with Turbo DNA-free kit (Ambion/Life Technologies (Carlsbad, Calif., USA)) to remove genomic DNA contamination or with RNA extraction kit (Qiagen (Valencia, Calif., USA)). Total RNA (100 ng to 1 μg) was used for reverse transcription reaction with SuperScriptIII (Life Technologies (Carlsbad, Calif., USA)) and random hexamer primers, according to the manufacturer's instructions. TaqMan probes and TaqMan Gene Expression Master Mix (Life Technologies (Carlsbad, Calif., USA)) were used for examining expression levels of the genes analyzed. The reverted cell lines expressed pluripotent marker genes but repressed differentiation marker genes at similar levels as those in mESCs (FIG. 4, panel D).

To determine whether the GFP⁺CD31⁺ cells were pluripotent, we performed blastocyst injections. In contrast to the parental Xi-GFP mEpiSCs, which did not produce chimeras, 70% of blastocysts injected with GFP⁺CD31⁺ cells produced chimeric mice (FIG. 4, panel E). The protocols for blastocyst injection and F1 mouse production were approved by the Institutional Animal Care and Use Committee at University of California, San Francisco. Single cells of GFP⁺CD31⁺ cells or mKate expressing Xi-GFP mEpiSCs were injected into blastocysts obtained from C57BL/6 mice, and GFP or mKate fluorescence was used for examining embryos' chimerism at embryonic day 14.5. Highly chimera F0 female mice were crossed with C57BL/6 male mouse to obtain the F1 offspring. This result suggested that the reverted cells readily colonize and contribute to mouse embryos at a high frequency, in sharp contrast to parental X-GFP mEpiSCs (FIG. 4, panel E).

Germ line transmission of the reverted cells was also observed (FIG. 4, panel F). Taken together, these results suggest that the reverted cells are XaXa and have differentiation ability equivalent to naïve pluripotent stem cells. Thus, the medium containing all of the components efficiently not only reactivates the Xi but also reverts primed cells to naïve cells.

### Example 5. LBAO Orchestrates the Transcription Factors Toward Naïve Pluripotency

To determine the timing of establishment of the naïve pluripotency gene expression pattern, reverse transcription and quantitative polymerase chain reaction (RT-qPCR) was used to examine RNA levels of the key pluripotency transcription factors KLF2, KLF4, PRDM14 and NANOG (Gillich et al., (2012) Cell Stem Cell 10(4):425-429; Guo et al., (2009) Development 136(7):1063-1069; Silva et al., (2009) Cell 138(4):722-737). Xi-GFP mEpiSCs were cultured in LBAO medium and RNA was collected every 24 hrs starting at day 2 of the culture. KLF2, KLF4, and PRDM14 were strongly up-regulated relative to untreated Xi-GFP mEpiSCs, however their kinetics differed (FIG. 5, panel A). KLF2 and KLF4 reached nearly maximal levels at day 2 (FIG. 5, panel A). PRDM14 was not induced until day 2-3 and reached maximal levels at day 4 (FIG. 5, panel A). In contrast, NANOG showed high expression in Xi-GFP mEpiSCs and showed a transient reduction at day 2 of treatment with LBAO (FIG. 5, panel A). Taken together, these data indicate that the LBAO medium robustly induces KLF2, KLF4, and PRDM14 expression. Since GFP⁺CD31⁺ cells emerged after day 5 (FIG. 5, panel B), the expression of transcription factors associated with naïve pluripotency preceded the appearance of CD31⁺GFP⁺ cells. Thus, the induction of the transcription factors may be a prerequisite for the conversion to naïve pluripotency.

In addition to acquiring LIF, BMP4, and LPA signaling, the switch from mEpiSC medium to LBAO medium involves the loss of signaling through bFGF and the ActivinA-SMAD2/3 axis. To determine whether it was the gain or loss of signaling that triggers the changes in KLF2, KLF4, and NANOG expression at early time points, the effect of transitioning cells to N2B27 (the basal medium for mEpiSC and LBAO media) without any additional factors (basal) was examined. Twenty-four hours after the medium change, KLF2 and KLF4, but not NANOG, are expressed at higher levels in LBAO and basal conditions than those in ActA+bFGF containing mEpiSC medium (FIG. 5, panel C). Since removing exogenous Activin A and bFGF from mEpiSC medium is sufficient to induce KLF2 and KLF4, these results suggest that Activin A and bFGF suppress KLF2 and KLF4 expression in mEpiSCs.

To determine the contribution of each component of LBAO to reactivation of Xi and regulation of naïve pluripotency transcription factor expression, the effects of removing each component individually was assessed. The % of GFP⁺ cells and expression levels of KLF2, KLF4, NANOG, and PRDM14 at day 6 were determined when LIF, BMP4, ascorbic acid, or OMPT were removed. There was a correlation between the efficiency of production of GFP⁺ cells and reactivation of transcription factors. Without LIF (BAO) almost no GFP⁺ cells arose and PRDM14 and KLF2 induction were substantially reduced (FIG. 5, panel D), consistent with a major role for LIF in establishing or maintaining the naïve state. Without OMPT (LBA), there was a slight reduction in the proportion of GFP⁺ cells and no significant change in transcription factor expression. Removing BMP4 (LAO) or ascorbic acid (LBO) reduced efficiency of GFP⁺ cells production to 40% of LBAO levels, with different effects of transcription factor expression (FIG. 5, panel D). While PRDM14 expression was reduced under both conditions, LBO showed a more dramatic decrease in NANOG expression while LAO was most affected for KLF4 expression. These results suggest that each transcription factor requires a specific combination of the components to achieve the LBAO expression levels that correlate with robust Xi-reactivation and naïve pluripotency.

To investigate whether KLF2, KLF4, or PRDM14 are required for the conversion to naïve cells, each of the transcription factors was depleted with shRNAs in Xi-GFP mEpiSCs and cultured the knockdown cells in LBAO medium for six days. GFP and the transcription factor STAT3, which lies downstream of LIF signaling, served as controls. While expressing shRNA against GFP reduced intensity of GFP fluorescence ten-fold, it did not affect the % of GFP⁺ cells compared with the mock control (FIG. 5, panel E and data not shown). Knock down of each factor resulted in a significant decrease in the proportion of GFP⁺ cells (FIG. 5, panel E), suggesting that KLF2, KLF4, and PRDM14 contribute to the efficient conversion of mEpiSCs to naïve cells.

Examination of the effects of STAT3, KLF2, KLF4, or PRDM14 knockdown on gene expression at day 6 of LBAO culture suggests that STAT3 lies upstream of the remaining factors (FIG. 5, panel F). STAT3 knock down decreased expression of Socs3, a direct target of LIF-STAT3 signaling, and all the transcription factors analyzed, with the exception of NANOG (FIG. 5, panel F), implicating the importance of LIF-STAT3 signaling pathway in the regulation of the transcription factors. Knock down of KLF2, KLF4, or PRDM14 resulted in very similar gene expression changes, with depletion of each factor significantly decreasing the others without affecting Stat3 or Socs3 (FIG. 5, panel F). Together these results suggest that the LIF-STAT3 pathways are up-stream of the KLF2-KLF4-PRDM14 circuit and there are no strong feedback loops between the LIF-STAT3 pathway and transcription factor circuit.

Inhibitor analysis implicated LPAR1/3 in the lipid signaling pathway (FIG. 3, panels D and E). To determine the relative contribution of these two receptors, each receptor was depleted in Xi-GFP mEpiSCs with shRNA and cells were cultured for six days in LBAO. Surprisingly LPAR1, but not LPAR3, depletion resulted in a substantial decrease in GFP⁺ cells (FIG. 5, panel E). In addition, LPAR1 knock down cells exhibited gene expression changes similar to those in the STAT3 depleted cells (FIG. 5, panel F), suggesting LPAR1 signaling intersects the LIF-STAT3 pathway. Since STAT3 knock down decreased LPAR1 expression (FIG. 5, panel F), a positive feedback loop between LIF and lipid signaling pathways may contribute to the conversion of mEpiSCs to naïve cells.

Together, these results show that LIF, BMP4, ascorbic acid and lysophosphatidic acid (LPA) are the factors that can induce Xi-reactivation, while, unexpectedly, Activin A and bFGF attenuate the LIF-induced Xi-reactivation. Here, a novel exogenous, gene-free system containing a mixture Xi-reactivation factors was developed that can efficiently convert primed PSCs to naïve PSCs. Using this system, an unanticipated role for lipid signaling in epigenetic regulation was discovered and the relationships between signaling pathways and endogenous KLF family and PRDM14 transcription factors which function during cellular reversion were uncovered. Thus, this system provides an unparalleled opportunity to elucidate endogenous molecular mechanisms and dynamic epigenetic changes involved in establishing naïve pluripotency.

### Example 6. The Combination of LIF, BMP4, Ascorbic Acid, and OMPT (LBAO) Efficiently Reactivate Xi and Convert Human Primed PSCs to Naïve PSCs

Human H9 hESCs and female iPSCs (collectively referred to as “the human PSCs”) are cultured in primed media for seven days, media replaced daily. The following day, the primed media is replaced with reversion media. The human PSCs are cultured in reversion media for 14 days. It is contemplated that by day 14 some of the human PSCs express two nascent transcript foci for both atrx and tsix in a nucleus, suggesting they are XaXa. In addition, it is contemplated that the reverted human PSCs express pluripotent marker genes but repress differentiation marker genes at similar levels as those in mESCs. Finally, it is contemplated that human-mouse chimera embryos are easily obtained following injection of the reverted human PSCs into mouse blastocysts in contrast to primed human PSCs as described in Gafni et al., (2013) Nature 504(7479):282-286 and Takashima et al., (2014) Cell 158(6):1254-1269.

### Example 7. Generation of Naïve Induced Pluripotent Stem Cells

Human fibroblasts are cultured in normal fibroblast medium (10% FBS in DMEM). After expansion of the fibroblasts, the fibroblasts are reprogrammed to iPSCs by nucleofecting episomal reprogramming vectors using the methods described by Okita et al. (2013) Stem Cells 31(3):458-466. A few days after nucleofection, in the fibroblast medium, nucleofected cells are transferred and cultured in naïve media for 3-4 weeks. It is contemplated that there is a 100-fold increase in reprogramming efficiency observed when fibroblasts are reprogrammed and maintained in naïve media versus conventional media. In addition, it is contemplated that the iPSCs derived in naïve media exhibit a more undifferentiated phenotype. These data suggest that LBAO acts as a booster of direct reprogramming, as well as to efficiently derive naïve iPSCs.

### Example 8. Generation of Naïve Induced Pluripotent Stem Cells from Established Human iPSCs Containing Episomal Reprogramming Factors

In contrast to mouse ESCs and iPSCs, most human ESCs and iPSCs are in a primed state, as assayed by XCI status, colony morphology, and marker gene expression, such as, KLF4, TFCP2L1 and TBX3. Since LPA treatment stimulated conversion from primed to naïve states in mouse pluripotent cells, it was next determined whether this bioactive lipid could promote naïve pluripotency in human cells. Conventional culture methods for human iPSCs (hiPSCs) maintain the hiPSCs in a pluripotent state by the addition of fibroblast growth factor (bFGF) and transforming growth factor beta (TGF-β), or activin A (ActA), to growth media and removal of these factors results in differentiation.

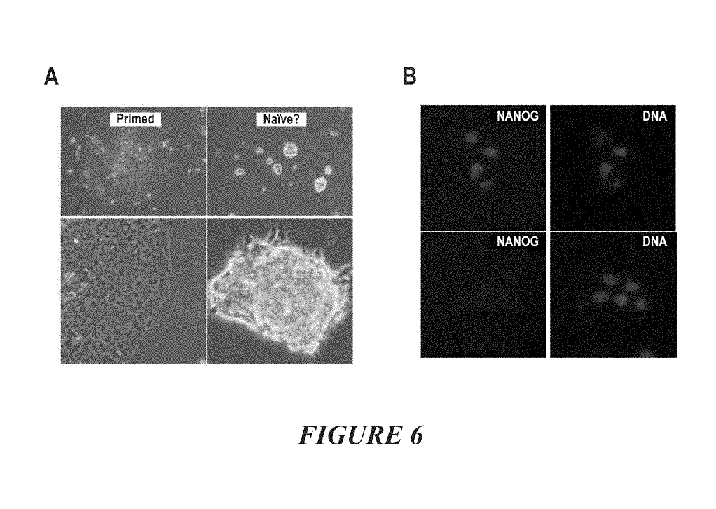

Here, episomal reprogramming factors (Oct3/4, SOX2, KLF4, 1-MYC, LIN28 and shRNA against p53 (as described in Okita et al. (2011) Nature Methods 8(5):409-412) were introduced into established, well-characterized hiPSCs to further reprogram to naive state. The hiPSCs were plated onto laminin-511 and allowed to expand to small colonies in primed media containing bFGF and TGF-β. The primed media was removed and replaced with reversion media 1 μM OMPT, 10 ng/mL LIF in StemFit basal media with no bFGF, TGF-β. Surprisingly, when bFGF and TGF-β were removed and the OMPT-containing media (reversion media) was added to hiPSCs containing the episomal reprogramming factors, cells with features of naïve pluripotency were observed. These naive-like cells formed three-dimensional dome shaped colonies characteristic of naïve pluripotent stem cells and very different from the colony morphology of primed hiPSCs. The naive-like cells in the LPA containing medium continue to express NANOG, suggesting that pluripotency has not been impaired (FIG. 6, panels A and B). These results suggest that LPA may also promote conversion to naive pluripotency in the human system. In addition, they provide the basis for further investigation of whether modulating the LPA-LPAR-RHO signaling axis can be used to generate and maintain human naïve pluripotent stem cells.

### Example 9. Generation of Naïve Induced Pluripotent Stem Cells from iPSCs with No Reprogramming Factors

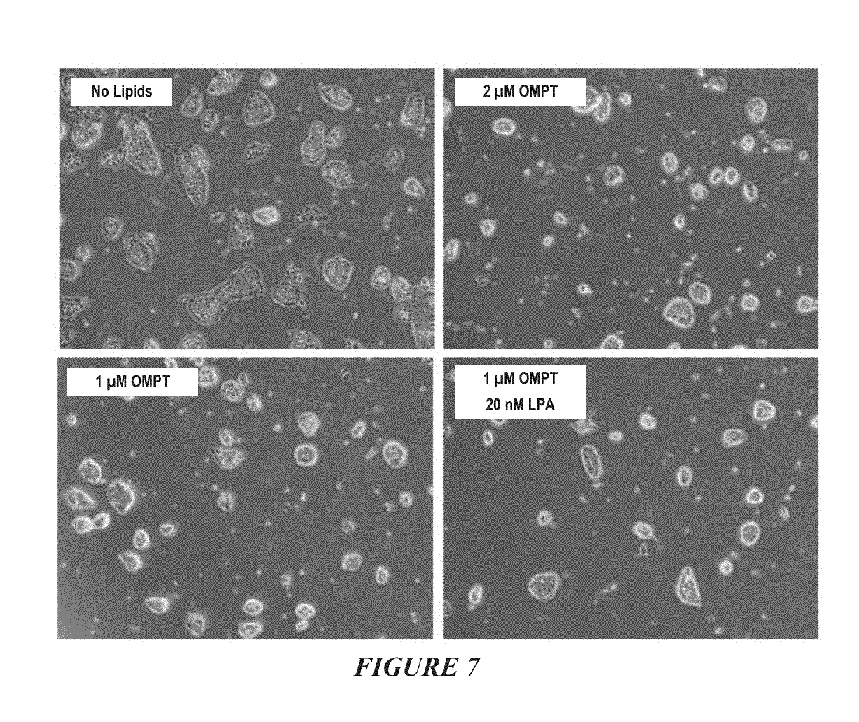

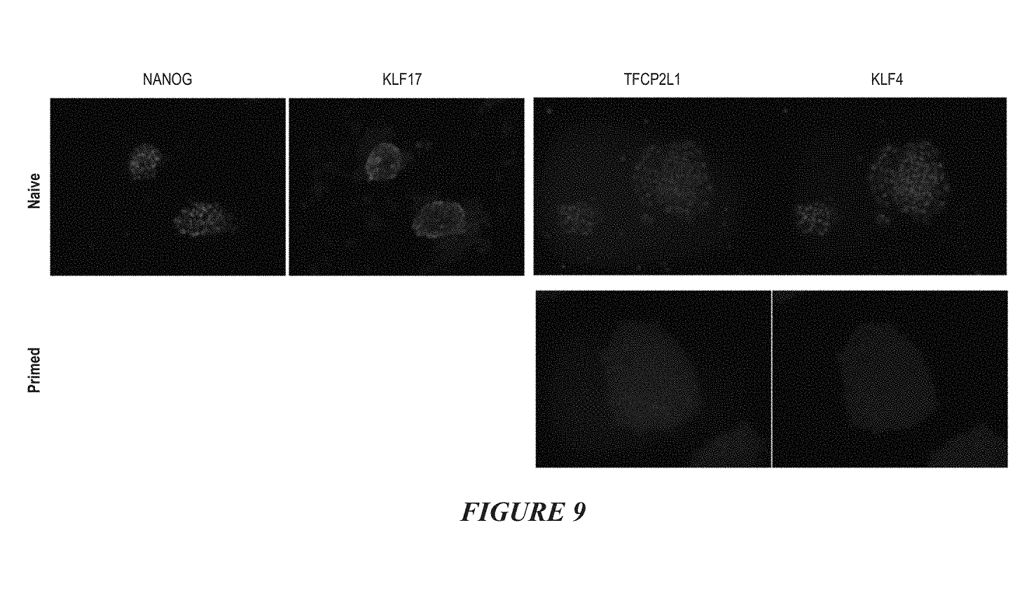

Human iPSCs were generated with episomal vectors expressing Oct4, Sox2, Klf4, myc, Lin28 and shRNA against p52 using the methods described by Okita et al. (2011) Nature Methods 8(5):409-412. The hiPSCs were grown in primed media containing bFGF and TGF-β in StemFit basal medium (or mTeSR orE8). Primed hiPSCs were then switched to reversion media and cultured on laminin-511 under hypoxic conditions. The reversion media contained 10 ng/mL LIF, 10 ng/mL Activin A, 100 ng/mL bFGF, 1 μM PD0325901 MEK inhibitor, 1 μM CHIR99021 GSK3β inhibitor, and 2 mM N-acetyl cysteine in StemFit basal medium with various concentrations of lipids (2 μM OMPT, 1 μM OMPT, 1 μM OMPT+20 nM LPA, or no lipids). After 5 to 7 days, three-dimensional dome shaped colonies, characteristic of naïve pluripotent stem cells, were observed. Human iPSCs grown in the presence of lipids (OMPT or OMPT/LPA) maintained the three-dimensional dome shaped colonies for more than one month (approximately five passages), while control cells cultured in the absence of lipids exhibited flat colonies, characteristic of primed pluripotent stem cells (FIG. 7). The naïve cells also expressed NANOG, KLF4, KLF17 and TFCP2L1, markers for naïve human pluripotent stem cells (FIG. 9). These data suggest that even in the absence of additional reprogramming factors, the lipids OMPT and LPA can revert primed hiPSCs to naïve hiPSCs and maintain a naïve pluripotent state.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

In addition, where the features or aspects of the invention are described in terms of Markush groups, those skilled in the art will recognize that the invention is also thereby described in terms of any individual member or subgroup members of the Markush group.

All publications, patent applications, patents and other references mentioned herein are expressly incorporated by reference in their entirety, to the same extent as if each were incorporated by reference individually. In case of conflict, the present specification, including definitions, will control.

1. A method for deriving a naïve pluripotent stem cell comprising: culturing a primed pluripotent stem cell in a culture media comprising an effective amount of an agonist of a lysophosphatidic acid receptor (LPAR), thereby reverting the primed pluripotent stem cell to a naïve pluripotent stem cell.

2. (canceled)

3. The method of claim 1, wherein the media further comprises an effective amount of one or more of a bone morphogenetic protein (BMP), an antioxidant, or a demethylase.

4. The method of claim 1, wherein the culture media further comprises an effective amount of a Signal Transducers and Activator of Transcription 3 (STAT3) activator.

5. The method of claim 4, wherein the STAT3 activator is leukemia inhibitor factor (LIF).

6. The method of claim 1, wherein the agonist of a LPAR is 1-oleoyl-2-methyl-sn-glycero-3-phosphothioate (OMPT), lysophosphatidic acid (LPA), or any combination thereof.

7. The method of claim 1, wherein the effective amount of the agonist of a LPAR is from about 10 nM to about 5 μM.

8. The method of claim 3, wherein the antioxidant is n-acetyl cysteine, and/or the demethylase is ascorbic acid.

9. (canceled)

10. The method of claim 1, wherein the culture media further comprises an effective amount of at least one of an ERK1/2 inhibitor, GSK3β inhibitor, a PKC inhibitor, activin A, transforming growth factor-β (TGF-β), basic fibroblast growth factor (bFGF), or any combination thereof.

11-13. (canceled)

14. The method of claim 1, further comprising culturing the primed pluripotent stem cells and/or naïve pluripotent stem cells on a fibronectin layer or a laminin layer.

15. The method of claim 1, wherein the primed pluripotent stem cell reverts to the naïve pluripotent stem cell in less than 10 days.

16. The method of claim 1, wherein the primed pluripotent stem cell expresses at least one exogenous reprogramming factor, at least one exogenous RNA, or both.

17-18. (canceled)

19. The method of claim 1, further comprising the step of culturing the primed pluripotent stem cell and/or the naïve pluripotent stem cell in hypoxic conditions for a period of time.

20-35. (canceled)

36. Cell culture media comprising 10 nM to about 10 μM of an agonist of a LPAR.

37. The cell culture media of claim 36, wherein the cell culture media further comprises an effective amount of one or more of a BMP, an antioxidant, a demethylase, or a STAT3 activator.

38. (canceled)

39. The cell culture media of claim 37, wherein the STAT3 activator is LIF.

40. The cell culture media of claim 36, wherein the agonist of a LPAR is OMPT, LPA, or any combination thereof.

41. The cell culture media of claim 36, wherein the agonist of a LPAR is present in the media at a concentration from about 10 nM to about 5 μM.

42. The cell culture media of claim 37, wherein the antioxidant is n-acetyl cysteine and/or the demethylase is ascorbic acid.

43. (canceled)

44. The cell culture media of claim 36, wherein the cell culture media further comprises an effective amount of an ERK1/2 inhibitor, GSK3β inhibitor, a PKC inhibitor, or any combination thereof.

45. (canceled)

46. A method for deriving naïve pluripotent stem cells comprising: culturing primed pluripotent stem cells in a culture media, wherein the culture media is substantially free of an ERK1/2 inhibitor, a GSK3β inhibitor, a PKC inhibitor, or any combination thereof.

47-55. (canceled)
